SEQUENCE LISTING (79 sequences)

SEQ ID NO: 1 - DNA, 30 bp, Artificial Sequence
  Synthetic Oligonucleotide
agtggggcctgcgctcgctgtccacacact

SEQ ID NO: 2 - DNA, 18 bp, Artificial Sequence
  Synthetic Oligonucleotide
gcctgcgctcgctgtcca

SEQ ID NO: 3 - DNA, 30 bp, Artificial Sequence
  Synthetic Oligonucleotide
cgtacatgaccacgcccagcccccaccagt

SEQ ID NO: 4 - DNA, 18 bp, Artificial Sequence
  Synthetic Oligonucleotide
tgaccacgcccagccccc

SEQ ID NO: 5 - DNA, 30 bp, Artificial Sequence
  Synthetic oligonucleotide
ggtcacgcggtgcttgggcttggccaggga

SEQ ID NO: 6 - DNA, 18 bp, Artificial Sequence
  Synthetic oligonucleotide
gcggtgcttgggcttggc

SEQ ID NO: 7 - DNA, 30 bp, Artificial Sequence
  Synthetic oligonucleotide
agcagaggcggtcgtgggtctggaaagagt

SEQ ID NO: 8 - DNA, 18 bp, Artificial Sequence
  Synthetic oligonucleotide
ggcggtcgtgggtctgga

SEQ ID NO: 9 - DNA, 30 bp, Artificial Sequence
  Synthetic oligonucleotide
tgacaaagcagaggcggtcgtgggtctgga

SEQ ID NO: 10 - DNA, 18 bp, Artificial Sequence
  Synthetic oligonucleotide
agcagaggcggtcgtggg

SEQ ID NO: 11 - DNA, 30 bp, Artificial Sequence
  Synthetic oligonucleotide
ttaatgtgcccgtccttgtccagcatgagg

SEQ ID NO: 12 - DNA, 18 bp, Artificial Sequence
  Synthetic oligonucleotide
tgcccgtccttgtccagc

SEQ ID NO: 13 - DNA, 30 bp, Artificial Sequence
  Synthetic oligonucleotide
accacgcccagcccccaccagtccactgca

SEQ ID NO: 14 - DNA, 18 bp, Artificial Sequence
  Synthetic oligonucleotide
cccagcccccaccagtcc

SEQ ID NO: 15 - DNA, 30 bp, Artificial Sequence
  Synthetic oligonucleotide
gcctcaggccgtgccgctggccgagtagga

SEQ ID NO: 16 - DNA, 18 bp, Artificial Sequence
  Synthetic oligonucleotide
ggccgtgccgctggccga

SEQ ID NO: 17 - DNA, 30 bp, Artificial Sequence
  Synthetic oligonucleotide
gtccagcgcagtccaccgccgcctcaggcc

SEQ ID NO: 18 - DNA, 18 bp, Artificial Sequence
  Synthetic oligonucleotide
cgcagtccaccgccgcct

SEQ ID NO: 19 - DNA, 30 bp, Artificial Sequence
  Synthetic oligonucleotide
tgctcactcgcggatgctggccgagtagga

SEQ ID NO: 20 - DNA, 18 bp, Artificial Sequence
  Synthetic oligonucleotide
ctcgcggatgctggccga

SEQ ID NO: 21 - DNA, 30 bp, Artificial Sequence
  Synthetic oligonucleotide
cgtacatgaccacacccagcccccaccagt

SEQ ID NO: 22 - DNA, 18 bp, Artificial Sequence
  Synthetic oligonucleotide
tgaccacacccagccccc

SEQ ID NO: 23 - DNA, 30 bp, Artificial Sequence
  Synthetic oligonucleotide
ggatggcccgcatccactcctccctctcgt

SEQ ID NO: 24 - DNA, 18 bp, Artificial Sequence
  Synthetic oligonucleotide
cccgcatccactcctccc

SEQ ID NO: 25 - DNA, 30 bp, Artificial Sequence
  Synthetic oligonucleotide
ctggggcccgctgcttgaggctgttggcga

SEQ ID NO: 26 - DNA, 18 bp, Artificial Sequence
  Synthetic oligonucleotide
cccgctgcttgaggctgt

SEQ ID NO: 27 - DNA, 30 bp, Artificial Sequence
  Synthetic oligonucleotide
agtccatggggtcctcgcctggggcccgct

SEQ ID NO: 28 - DNA, 18 bp, Artificial Sequence
  Synthetic oligonucleotide
tggggtcctcgcctgggg

SEQ ID NO: 29 - DNA, 30 bp, Artificial Sequence
  Synthetic oligonucleotide
tttagcccgtgccttgctgaccgccacttc

SEQ ID NO: 30 - DNA, 18 bp, Artificial Sequence
  Synthetic oligonucleotide
ccgtgccttgctgaccgc

SEQ ID NO: 31 - DNA, 30 bp, Artificial Sequence
  Synthetic oligonucleotide
agcacaggcggtcgtgggtctggaaggcat

SEQ ID NO: 32 - DNA, 18 bp, Artificial Sequence
  Synthetic oligonucleotide
ggcggtcgtgggtctgga

SEQ ID NO: 33 - DNA, 30 bp, Artificial Sequence
  Synthetic oligonucleotide
cacccagcccccaccagtccacggcccggc

SEQ ID NO: 34 - DNA, 18 bp, Artificial Sequence
  Synthetic oligonucleotide
gcccccaccagtccacgg

SEQ ID NO: 35 - DNA, 30 bp, Artificial Sequence
  Synthetic oligonucleotide
cgctgggccccccaccaagcctctgcttgg

SEQ ID NO: 36 - DNA, 18 bp, Artificial Sequence
  Synthetic oligonucleotide
gccccccaccaagcctct

SEQ ID NO: 37 - DNA, 30 bp, Artificial Sequence
  Synthetic oligonucleotide
agtgggtccgctggtccagctccagtaagc

SEQ ID NO: 38 - DNA, 18 bp, Artificial Sequence
  Synthetic oligonucleotide
tccgctggtccagctcca

SEQ ID NO: 39 - DNA, 30 bp, Artificial Sequence
  Synthetic oligonucleotide
cagcgagcgtgcgtcctctgcgtgggcaga

SEQ ID NO: 40 - DNA, 18 bp, Artificial Sequence
  Synthetic oligonucleotide
gcgtgcgtcctctgcgtg

SEQ ID NO: 41 - DNA, 30 bp, Artificial Sequence
  Synthetic oligonucleotide
acccagcggtgatggcagcgagcgtgcgtc

SEQ ID NO: 42 - DNA, 18 bp, Artificial Sequence
  Synthetic oligonucleotide
cggtgatggcagcgagcg

SEQ ID NO: 43 - DNA, 30 bp, Artificial Sequence
  Synthetic oligonucleotide
cccctaggccccaccagtctactgctcggc

SEQ ID NO: 44 - DNA, 18 bp, Artificial Sequence
  Synthetic oligonucleotide
ggccccaccagtctactg

SEQ ID NO: 45 - DNA, 30 bp, Artificial Sequence
  Synthetic oligonucleotide
aaatggaaaaacagctcgcccccattaaca

SEQ ID NO: 46 - DNA, 18 bp, Artificial Sequence
  Synthetic oligonucleotide
aaaaacagctcgccccca

SEQ ID NO: 47 - DNA, 30 bp, Artificial Sequence
  Synthetic oligonucleotide
catacatgacaacccctaggccccaccagt

SEQ ID NO: 48 - DNA, 18 bp, Artificial Sequence
  Synthetic oligonucleotide
tgacaacccctaggcccc

SEQ ID NO: 49 - DNA, 30 bp, Artificial Sequence
  Synthetic oligonucleotide
aatgcggccgcctctcattgtccatgcagt

SEQ ID NO: 50 - DNA, 18 bp, Artificial Sequence
  Synthetic oligonucleotide
gccgcctctcattgtcca

SEQ ID NO: 51 - DNA, 30 bp, Artificial Sequence
  Synthetic oligonucleotide
gacttattctcgtccacttgcagagtagga

SEQ ID NO: 52 - DNA, 18 bp, Artificial Sequence
  Synthetic oligonucleotide
ttctcgtccacttgcaga

SEQ ID NO: 53 - DNA, 30 bp, Artificial Sequence
  Synthetic oligonucleotide
gtgaaaggtggcgaggggtgaggacccttg

SEQ ID NO: 54 - DNA, 18 bp, Artificial Sequence
  Synthetic oligonucleotide
ggtggcgaggggtgagga

SEQ ID NO: 55 - DNA, 30 bp, Artificial Sequence
  Synthetic oligonucleotide
ggatggcggttgtccactcctcccgctcct

SEQ ID NO: 56 - DNA, 18 bp, Artificial Sequence
  Synthetic oligonucleotide
cggttgtccactcctccc

SEQ ID NO: 57 - DNA, 30 bp, Artificial Sequence
  Synthetic oligonucleotide
cctcctcctgcttcttgaggccgtcagcca

SEQ ID NO: 58 - DNA, 18 bp, Artificial Sequence
  Synthetic oligonucleotide
cctgcttcttgaggccgt

SEQ ID NO: 59 - DNA, 30 bp, Artificial Sequence
  Synthetic oligonucleotide
aggtggaagaacagctcgcccccgttggcg

SEQ ID NO: 60 - DNA, 18 bp, Artificial Sequence
  Synthetic oligonucleotide
aagaacagctcgcccccg

SEQ ID NO: 61 - DNA, 30 bp, Artificial Sequence
  Synthetic oligonucleotide
cctcggagcccccgccaagcctctgcttgg

SEQ ID NO: 62 - DNA, 18 bp, Artificial Sequence
  Synthetic oligonucleotide
agcccccgccaagcctct

SEQ ID NO: 63 - DNA, 30 bp, Artificial Sequence
  Synthetic oligonucleotide
aggtggaagaacagctcacccccgttggca

SEQ ID NO: 64 - DNA, 18 bp, Artificial Sequence
  Synthetic oligonucleotide
aagaacagctcacccccg

SEQ ID NO: 65 - DNA, 30 bp, Artificial Sequence
  Synthetic oligonucleotide
ttgatgtggccatctttgtccagcatgagg

SEQ ID NO: 66 - DNA, 18 bp, Artificial Sequence
  Synthetic oligonucleotide
tggccatctttgtccagc

SEQ ID NO: 67 - DNA, 30 bp, Artificial Sequence
  Synthetic oligonucleotide
ggatagcttctgtccattcttccctttcct

SEQ ID NO: 68 - DNA, 18 bp, Artificial Sequence
  Synthetic oligonucleotide
cttctgtccattcttccc

SEQ ID NO: 69 - DNA, 30 bp, Artificial Sequence
  Synthetic oligonucleotide
cctcttcttgcctctgcagtctgtctgcta

SEQ ID NO: 70 - DNA, 18 bp, Artificial Sequence
  Synthetic oligonucleotide
cttgcctctgcagtctgt

SEQ ID NO: 71 - DNA, 30 bp, Artificial Sequence
  Synthetic oligonucleotide
tttatgtggccatctttgtccagcattaga

SEQ ID NO: 72 - DNA, 18 bp, Artificial Sequence
  Synthetic oligonucleotide
tggccatctttgtccagc

SEQ ID NO: 73 - DNA, 30 bp, Artificial Sequence
  Synthetic oligonucleotide
catctggtcctccaccaaggcgtttatttg

SEQ ID NO: 74 - DNA, 18 bp, Artificial Sequence
  Synthetic oligonucleotide
gtcctccaccaaggcgtt

SEQ ID NO: 75 - DNA, 18 bp, Artificial Sequence
  Synthetic oligonucleotide
gactcctctcaatgttca

SEQ ID NO: 76 - DNA, 18 bp, Artificial Sequence
  Synthetic oligonucleotide
ctattggatgatggatga

SEQ ID NO: 77 - DNA, 18 bp, Artificial Sequence
  Synthetic oligonucleotide
agcaccaacaatcaacgg

SEQ ID NO: 78 - DNA, 18 bp, Artificial Sequence
  Synthetic oligonucleotide
tgaacaagcacagcaccc

SEQ ID NO: 79 - DNA, 18 bp, Artificial Sequence
  Synthetic oligonucleotide
tgaacaaacacagcaccc

(19) United States
(12) Patent Application Publication
(10) Pub. No.: US 2009/0239932 A1
(43) Pub. Date: Sep. 24, 2009

(54) Antisense oligonucleotides against protein kinase isoforms alpha, beta and gamma

(76) Inventors: Graham Sale, Southampton (GB); Elizabeth Sale, Southampton (GB); Conrad Hodgkinson, Southampton (GB); Neil P. Jones, Twyford (GB)

Correspondence Address: NIXON & VANDERHYE, PC, 901 NORTH GLEBE ROAD, 11TH FLOOR, ARLINGTON, VA, 22203, US

(21) Appl. No.: 11/990,655

(22) PCT Filed: Aug. 18, 2006
(86) PCT No.: PCT/GB2006/003100

§ 371 Date: Jan. 14, 2009

(30) Foreign Application Priority Data
Aug. 18, 2005 (GB) 0516967.7

(51) Int. Cl. A61K 31/7088; C07H 21/04; C12N 15/63; C12N 5/10

(52) U.S. Cl. 514/44 A; 536/24.5; 435/320.1; 435/325; 435/375

(57) ABSTRACT

Antisense compounds are provided for inhibiting PKB (protein kinase B) alpha, beta and gamma. The antisense compounds display high potency and selectivity. The antisense compounds do not suffer from problems of dimerisation or self-hybridization and have also been selected to not affect other enzymes. The antisense compounds may be used singularly or in combination to inhibit one, two or all of PKB alpha, beta and gamma and hence treat conditions in which these enzymes are important.

### BACKGROUND OF THE INVENTION

Insulin and other growth factors promote a variety of key biological responses, including increased glucose uptake into cells, stimulation of glycogen synthesis, DNA synthesis, protein synthesis, cell growth, differentiation and cell survival. Recent studies have highlighted the importance of the phosphatidylinositol 3-kinase/protein kinase B (PI3K/PKB) pathway in regulating many of these key cellular processes. PKB is a major signalling intermediate in this pathway and is a serine/threonine kinase, which exists in three isoforms, PKBα, PKBβ and PKBγ. PKB also appears to be a crucial pro-survival kinase.

PKB acts downstream of PI3K and is activated through recruitment to the plasma membrane by PI3K lipid products and by phosphorylation at two regulatory sites, one in the kinase domain (Thr308 for PKBα) and the other in the C-terminal domain (Ser473 for PKBα). The kinase that phosphorylates Thr308 has been identified as 3-phosphoinositide-dependent protein kinase-1 (PDK1). A distinct Ser473 kinase (PDK2) exists but has not been identified.

One target of PKB is glycogen synthase kinase-3 (GSK-3) which phosphorylates and inactivates glycogen synthase. However, many studies now demonstrate that GSK-3 plays a more diverse role and acts as a critical downstream regulatory switch for a divergent array of responses from multiple stimuli, which when dysregulated, has been implicated in diseases such as diabetes, cancer, Huntington's disease, Alzheimer's and bipolar disorder.

The PI3K/PKB pathway also plays a critical role in cancer and is activated in a range of tumours. For example: i) PI3K is amplified and overexpressed in ovarian and cervical cancers; ii) PKB is overexpressed and activated in breast, ovarian, pancreatic, prostate and stomach malignant cancers; iii) the upstream regulators of the PI3K/PKB pathway, e.g. EGFR and ErbB2, are mutated, amplified and overexpressed in ovarian, lung and breast cancer while another upstream regulator, Ras, is frequently mutated in pancreatic and colorectal cancers; and iv) PTEN, which encodes a phosphatidylinositol (PI) lipid phosphatase, is frequently mutated and deregulated in glioblastoma, prostate and endometrial cancers and also in melanomas. Additionally integrin-linked kinase (ILK), which can phosphorylate PKB, has been shown to be elevated in melanomas and cancers of the breast, prostate, stomach and colon, while overexpression of ILK in nude mice results in tumorigenicity.

These findings, together with many other results using mouse genetic models, strongly validate the PI3K/PKB pathway as a key target for drug development for cancer and the control of other diseases. Recent research indicates that activation of the PI3K/PKB pathway also plays an important role in the development of tumour cell resistance to both chemotherapy and radiation, thereby further highlighting the need for therapeutic inhibition of this pathway in these disease states.

To date, small-molecule inhibitors of PI3K or PKB have not been developed for clinical trials. This is due to a number of problems including specificity issues.

### BRIEF DESCRIPTION OF THE FIGURES

FIG. 1 shows specific depletion of both PKBα and PKBβ by double AS(αβ) and PKBγ by single AS(γ). 3T3-L1 adipocytes were treated with the indicated oligonucleotides, as detailed in Table 10. After 120 hours cells were extracted by scraping and Western blots performed to determine the expression of, from top to bottom, PKBα (A), PKBβ (B), PKBγ (C), PDK1 (D), PKC (E), and p70S6kinase (F). PKC was blotted with phospho-(Thr) PDK1 substrate antibody. The 80 Kda PKC band is shown. Oligonucleotides concentrations were 5 μM except for single AS(γ) and single MM(α), which were 10 μM. Co, Control untreated cells; CL, Lipofectamine 2000™-only treated cells.

FIG. 2 shows dose response curves for depletion of PKBα and PKBβ by double AS(αβ) and PKBγ by single AS(γ). Panels (A and B). 3T3-L1 adipocytes were treated with the double AS(αβ) oligonucleotides as detailed in Table 5. The concentration of each antisense probe in the medium is indicated. Cells were extracted after 120 hours by scraping and PKBα (A) and PKBβ (B) expression determined by Western blotting and densitometric scanning. Results are expressed as a % of Lipofectamine 2000™-only controls and are mean±SEM for three independent determinations.

FIG. 3 shows that PKB knock down is associated with loss of insulin-stimulated PKB activity. 3T3-L1 adipocytes were treated with or without oligonucleotides for 120 hours. Cells were then stimulated with or without 100 nM insulin for 5 minutes and whole cell extracts used for determination of PKB activity and analysis by Western blotting. (A) Insulin-stimulated PKB activity. (B) Western blot of total cell PKB. Oligonucleotides concentrations were 5 μM except for single AS(γ) and single MM(γ), which were 10 μM. Insulin-stimulated PKB activity is expressed as the fold increase in activity relative to that of unstimulated untreated control cells. Results are of a representative experiment. Panel (C) shows the effect a triple antisense α, β and γ set of antisense compounds on insulin stimulated PKB Ser⁴⁷³P compared to Lipofectamine 2000™ only treated cells and a mismatched set of oligonucleotides.

FIG. 4 shows the effect of a triple antisense combination against PKBα, β and γ, a triple mismatch control and a Lipfectamine 2000™ only treated cell control on insulin stimulated WNK1Thr⁶⁰ (top), ATP citrate lyase Ser⁴⁵⁴ (middle) and Tuberin Thr¹⁴⁶² (bottom) phosphorylation.

FIG. 5 shows a time course for the number of viable human breast cancer cells in a culture following treatment with Triple AS (α,β,γ) antisense oligonucleotides in comparison to a control culture treated solely with the transfection agent Lipofectamine.

FIG. 6 shows: (A) Western blot showing the level of PKB-α following antisense treatment with the indicated oligonucleotides; (B) Western blots showing the level of PKB-β with further antisense oligonucleotides against PKBβ; and (C) also Western blots showing the level of PKBγ with further antisense oligonucleotides against PKBγ.

### SUMMARY OF THE INVENTION

The present invention provides potent and highly selective antisense compounds against PKB (protein kinase B) α, β and γ. As well as being at least as potent as any of the previously identified antisense compounds against PKB, the compounds of the invention have a number of other advantages including typically having high selectivity so that they only inhibit the intended PKB enzyme and also that they typically display an inability to form duplexes and/or to fold back on themselves which would be likely to impair their usefulness. In addition, the compounds target regions of PKB which do not form hairpins likely to interfere with antisense activity.

Accordingly, the present invention provides an antisense compound 8 to 30 nucleotides in length which is capable of inhibiting wholly or partially the activity of Protein Kinase B (PKB) isoforms α, β and/or γ, the antisense compound comprising the sequence of:
(i) any one of sequences of SEQ ID Nos: 1 to 74;
(ii) the equivalent region to the sequence of SEQ ID Nos: 1 to 74 in a non-human mammalian PKB; or
(iii) a sequence with at least 80% sequence identity to the sequence of (i) or (ii).

In one particularly preferred embodiment the antisense compound comprising the sequence of:
(i) any one of sequences of SEQ ID Nos: 1 to 54;
(ii) the equivalent region to the sequence of SEQ ID Nos: 1 to 54 in a non-human mammalian PKB; or
(iii) a sequence with at least 80% sequence identity to the sequence of (i) or (ii).

The present invention also provides a combination comprising an antisense compound according to the invention and at least one other antisense compound.

The invention also provides a polynucleotide capable of expressing an antisense compound according to the invention or a combination according to the invention. The invention also provides for a host cell comprising a polynucleotide of the invention. The invention also provides a cell comprising an antisense compound of the invention.

The invention also provides an antisense compound of the invention, a combination of the invention, a polynucleotide of the invention or a host cell of the invention for use in a method of treatment of the human or animal body by therapy.

The invention additionally provides for the use of an antisense compound of the invention, a combination according to the invention, a polynucleotide according to the invention or a host cell according to the invention in the manufacture of a medicament for treating cancer, diabetes, a degenerative neurological condition and/or a mental disorder.

In another instance the invention also provides products comprising:

(i) an antisense compound of the invention or a combination of the invention; and

(ii) a therapeutic agent other than (i)

for the simultaneous, separate or sequential use in the treatment of cancer, diabetes, a degenerative neurological condition and/or a mental disorder. Polynucleotides and cells of the invention may also be used in combination with (ii).

The present invention also provides a method of treating cancer, diabetes, a degenerative neurological condition and/or a mental disorder which comprises administering an effective amount of an antisense compound of the invention, a combination of the invention, a polynucleotide of the invention or a host cell of the invention to a subject with such a disorder.

The invention also provides for a method of inhibiting wholly or partially the expression of PKB α, β and/or γ in cells or tissues comprising contacting said cells or tissues with an antisense compound of the invention, a combination of the invention, a polynucleotide of the invention or a host cell of the invention. The method may be applied in vivo, in vitro or ex vivo.

### DETAILED DESCRIPTION OF THE INVENTION

In any of the instances herein where reference is made to something “comprising” specific elements, in one instance it may consist essentially of such elements or in particular consist of such elements.

The present invention employs an alternative approach to small molecule inhibition of PKB and in particular employs antisense compounds for the therapeutic targeting of the PKB pathway in tumours and other diseases. The compounds are typically short antisense synthetic oligonucleotides which are specific for sequences in the mRNA for the protein. Knockdown of PKB protein as a method of cancer therapy is especially effective because levels of activated PKB (phosphorylated on Ser 473 [pSer473-PKB]) have been found to correlate with prognosis in patients with a wide range of cancers. Therefore, by modulating PKB protein in the cell, and consequently levels of pSer473-PKB, it is possible to regulate cancer outcome. The antisense strategy may also be used to treat the other disorders PKB is important in.

Individual PKB isoforms, PKBα, PKBβ and PKBγ, may have unique as well as common functions within the cell. For example, relative to PKBα and PKBγ, PKBβ has been shown to play the predominant role in phosphorylating and inactivating GSK-3 in response to insulin. PKBβ may also act as the major isoform that signals increased insulin-stimulated glucose transport. Specific isoforms of PKB are also selectively elevated in numerous cancer types. PKBα activity is increased in prostate or breast cancers, while PKBβ amplification occurs in some pancreatic or ovarian cancers. PKBγ has been reported to be elevated in many melanomas.

Thus, as the invention provides antisense compounds against all three PKBs the invention may be used to selectively inhibit one, two, or alternatively all three PKB isoforms simultaneously. It is possible to tailor therapy regimes to the individual patient. This has the advantage of minimising the concentration of drug required without compromising on drug efficacy. Conditions mentioned herein mentioned as involving a particular PKB may be treated by specifically modulating that PKB in some instances.

Antisense compounds of the invention and in particular the phosphorothioate probes of the present invention are designed to contain specific characteristics that optimise antisense performance. These features help in drug development. Firstly, the sequences are preferably short (in particular 18mers may be employed and represent a preferred length). The advantage of such lengths is that the antisense compounds are easily taken up into most cells, while at the same time being sufficiently long to confer sequence specific hybridisation with the target alone. Secondly, the antisense compounds are preferably selected to contain high G/C base content and high Tm of binding to the target, which increases potency of the antisense compounds and causes the greatest maximal inhibition of expression of the target protein within the cell. Thirdly, antisense compounds are preferably designed to bind to regions on the target DNA that are free of potential hairpins, which could otherwise interfere with antisense effect. Finally, antisense compounds are preferably selected to contain ideally no, or alternatively minimal, ability to form duplexes or to fold back on themselves forming a small hairpin, which could otherwise impede uptake into tissues or cells and/or reduce efficacy. In a highly preferred instance of the invention the antisense compounds employed have at least one of, preferably at least two of, more preferably at least three of and even more preferably at least four of these features. The compound may, in some cases, display all of the above properties.

Antisense compounds are used as tools to investigate signalling pathways or as drugs to treat diseases. However, relatively few antisense compounds are suitable for drug development. This is because drugs preferably satisfy a number of stringent criteria, in particular they preferably show both high potency and high specificity for the desired target. Drugs that are potent allow lower doses to be administered so that side effects are kept to an absolute minimum and hence are clearly advantageous over those that do not.

The PKB antisense compounds of the invention hybridise to DNA with high Tm and are more potent in vivo than almost all of those described previously. Those few antisense compounds with similar potency to those of the invention lack the other desirable features of the antisense compounds of the invention.

These features are important if an antisense compound is to be used as a drug. For example, an antisense compound that strongly hybridises with itself, either by intramolecular or intermolecular bonding, possesses an undesirable characteristic, which could interfere with efficacy during treatment and thereby limit development of the antisense compound as a useful drug. The advantage of the present invention is that antisense compounds described typically show no, or lower potential, to dimerise and/or fold-back on themselves than those described previously which displayed similar potency. As an illustration of this, the oligonucleotides DASα and DASβ are completely unable to dimerise or fold back on themselves at all i.e. they are free strands in solution and are highly potent and are preferred antisense compounds of the invention. The antisense compounds referred to previously in the art with similar potency to those of the invention both dimerise reducing their efficacy substantially in comparison to the antisense compounds of the invention.

The specificity of an inhibitor is highly important if it to be developed as a successful therapeutic drug. Any antisense compound that significantly depletes other important cell signalling intermediates in the cell cannot usually be used for therapy. An advantage of the present invention is that the specificity of the antisense regions targeted have been tested and they have been confirmed as being highly specific. Prior art antisense compounds have not been assessed for specificity and analysis indicates that those few compounds with potency similar to those of the invention are likely to display a degree of cross-reactivity against other important kinases casting doubts over their therapeutic usefulness. Other antisense compounds in the art contain modifications likely to trigger the immune system in a non-specific way rendering them less desirable.

The specificity of particular antisense compounds of the invention has been assessed in a number of ways. Firstly, the antisense compounds were preferably designed to be specific and were selected on the basis that they did not bind to any other sequences in the database. Importantly, kinases such as PDK1, SGK isoforms, p70S6kinase, PKCλ, PKCζ and/or MAP kinase isoforms do not typically contain sequences which will bind the probes and preferably an antisense compound of the invention will not inhibit such kinases. Secondly, specificity of the antisense effect has been tested in vivo. Treatment of cells using antisense compounds that target the PKB antisense regions in the invention does not typically alter the amounts of other key components upstream and downstream of PKB as determined by Western blots. In particular, cellular levels of PDK1, PKC, p70S6kinase or 4E-BP1 are typically unaffected by PKB antisense antisense compound treatment. Thirdly, the PKB antisense compounds do not typically affect other major proteins in the cell as assessed by coomassie blue staining of proteins separated by gel electrophoresis and transferred to nitrocellulose. Fourthly, control phosphorothioate antisense compounds in the form of mismatch antisense compounds (consisting of the antisense sequence with base changes along the length of the probe) do not significantly affect the levels of PKBα, PKBβ, or PKBγ within cells. Fifthly, the expression of extracellular-signal regulated kinase (ERK1/2) and signalling of events that occur through the ERK1/2 pathway was normal showing that this parallel pathway was unperturbed. Lastly, the antisense treatment typically has no effect on signalling responses that occur independently of PKB, showing that there was no general impairment of cell function.

The Examples of the present invention demonstrate that the PKB antisense compounds can also be used in combination in cells to potently knockdown one, two, or all three PKB isoforms simultaneously. Treatment of diseases with PKB antisense inhibitors in combination can confer significant advantages. Combination therapy using two or more antisense compounds that target different PKB isoforms can be used to increase drug potency. Multiple antisense compounds may be used against the same PKB isoform to increase inhibition still further.

Combination treatment using antisense compounds that target two or more different PKB isoforms is especially beneficial where all, or just two, PKB isoforms are dysregulated in the diseased state. In a further instance, PKB antisense can be used to enhance the action of other drugs, including traditional chemotherapy and radiotherapy treatments for cancer. This approach is particularly preferred and offers the advantages of increased therapeutic effectiveness and, by lowering the drug doses used, reduces financial cost and risk of undesirable side effects.

The Examples of the present application further demonstrate the utility of the invention by showing that PKB antisense treatment of cells can abrogate the phosphorylation of key proteins that act as downstream PKB substrates, including GSK-3, WNK-1, ATP citrate lyase and the tumour suppressor protein, Tuberin. Thus, importantly, the antisense compounds are able to modulate signalling pathways that play central roles in tumour development and other disease states and hence can be used to treat such conditions. The compounds of the invention may be used to modulate phosphorylation of such proteins and to treat such conditions. In one instance, the phosphorylation, and its inhibition, of one or more, two, or all of GSK-3, WNK-1, ATP citrate lyase may be measured in assessing an antisense compound of the invention.

Cancer is a major killer worldwide and accounts for 6 million deaths per year. Carcinogenesis results from the imbalance between cell growth and division on the one hand and programmed cell death (apoptosis) on the other. Current conventional treatments such a chemotherapy and radiation are in many cases inadequate, with unacceptable side effects. New rationally designed anti-cancer drugs are just emerging onto the market. However, there is substantial scope for additional novel drugs, which are specifically designed to the individual biochemical characteristics of each cancer. Thus, PKB antisense compounds of the invention can be applied for the treatment of cancer and in particular a range of major cancers e.g. breast, lung, prostate, melanoma, pancreatic and stomach cancer, including cancers that have become resistant to conventional therapies. Local application of PKB antisense by cream, enema or inhalation can be used in the treatment of a range of cancers such as melanomas, colon or lung cancer. The present invention provides such formulations comprising an antisense compound of the invention. Antisense may be particularly effective as part of a combination therapy regime by increasing the sensitivity of cancers to other anti-cancer drugs or treatment.

Thus, the invention provides compositions comprising an antisense compound of the invention and a further therapeutic agent, The antisense compounds of the invention may also be administered simulataneously, separately, or sequentially with each other or with other further therapeutic agents. Possible further therapeutic agents include any mentioned herein and include antisense compounds against other genes including, for instance, any mentioned herein. The antisense compounds of the invention may be used in combination with other chemotherapy and/or radiation therapy to treat cancer, including any of those mentioned herein.

Huntington's disease, Alzheimer's, bipolar disorder and diabetes are also characterised by abnormalities in PI3K/PKB signalling pathway and the antisense compounds of the invention may be used to treat such conditions. Regulation of the level of one or more PKB isoforms by antisense therapy can confer benefits in these and other conditions. Huntington's disease is characterized by choreiform movements, psychiatric and cognitive decline resulting from graded loss of medium spiny projection neurons in the striatum. The antisense compounds of the invention may prevent or ameliorate such symptoms. Early events in the disease cascade, which predate overt pathology, include the activation of the PKB pro-survival signalling pathway via phosphorylation of GSK-3.

In Alzheimer's disease, PKB may protect against neuronal cell death. Significant increases in the levels of phosphorylation of PKB substrates, including GSK-3βSer9, tauSer214, mTORSer2448, and decreased levels of the PKB target, p27kip1, have been found in Alzheimer's temporal cortex. Dysregulation of GSK-3β may contribute to the pathophysiology of bipolar disorder. The antisense compounds of the invention may be used to treat Alzheimer's and help return levels of such indicators back to normal.

Studies focusing on PKB in diabetes suggest both decrease and increase of PKB activity in diabetes mellitus, depending on the tissues and cells and clinical contexts. Atypical PKCs, ζ/λ, may also play a role in mediating signals to targets that are downstream of PKB, for example by phosphorylating GSK-3, and thus could be important in disease progression. Therapy regimes that regulate the activities of PKB isoforms and atypical PKCs simultaneously may confer significant advantages and the invention provides for such regimes. The invention provides compositions comprising an antisense compound of the invention and a modulator of atypical PKCs, including, for instance, any of those mentioned herein. The modulators of atypical PKCs may also be antisense compounds and may, for instance, have any of the properties of the PKB antisense compounds mentioned herein, but be targeted against PKC.

The targeting of signalling intermediates critical in tumour cell development and in other diseases by antisense knockdown may provide additional benefit.

### Antisense Compounds

The invention provides antisense compounds against PKB α, β and/or γ. The invention may be used to inhibit PKB α, β and/or γ Thus, in particular, the invention provides an antisense compound 8 to 30 nucleotides in length which is capable of inhibiting wholly or partially the activity of Protein Kinase B (PKB) isoforms α, β and/or γ, the antisense compound comprising the sequence of:
(i) any one of the sequences of SEQ ID Nos: 1 to 74;
(ii) the equivalent region to the sequence of SEQ ID Nos: 1 to 74 in a non-human mammalian PKB; or
(iii) a sequence with at least 80% sequence identity to the sequence of (i) or (ii).

The non-human mammalian PKB may be from any of the animals mentioned herein and may be their PKB α, β and/or γ. In an especially preferred embodiment the antisense compound comprises the sequence of

(i) any one of sequences of SEQ ID Nos: 1 to 74;

(ii) a sequence with at least 80% to the sequence of (i) or (ii).

In one especially preferred embodiment, the antisense compound comprises the sequence of any one of SEQ ID Nos: 1 to 54, the equivalent sequence in a non-human mammalian PKB, or a sequence with at least 80% sequence identity to any of the preceding.

The antisense compound may in some instances be 28 or less, preferably 26 or less, even more preferably 24 or less, still more preferably 22 or less and more preferably 20 or less nucleotides in length. In some instances the compound may be 25 or less nucleotides in length. In some instances the compound may be 20 or less nucleotides in length. In some instances, the compound may be at least 10, preferably at least 12, more preferably at least 14, even more preferably at least 16 and still more preferably at least 18 nucleotides in length. In particular instances, the length of the compound may be from any of the minimum lengths mentioned up to any of the maximum lengths mentioned above. In particularly preferred instances the compound may be from 15 to 22, preferably from 17 to 21 nucleotides and even more preferably be a 18 mer. In one preferred instance, the antisense compound may be less than 29 bases in length. In the instances of specific sequences indicated herein which are longer than those lengths specified above, shorter regions of those sequences of the specified lengths above may be employed, as may the equivalent regions from non-human mammalian PKBs or sequences with one of the levels of sequence identity specified herein.

In the instance of SEQ ID Nos: 1 and 3 in a preferred instance shorter lengths may be employed, for instance 25 or less, preferably 23 or less, more preferably 21 or less and still more preferably 19 or less bases in length.

The level of sequence identity to any of the sequences of SEQ ID NOS: 1 to 74 may be at least 80%, still more preferably at least 85%, and even more preferably at least 90%. In some instances, the level of sequence identity may be at least 95% and in an especially preferred embodiment the level of sequence identity may be 100%. In some instances the level of identity may be at least 83%, preferably at least 88% and more preferably at least 94%. The level of sequence identity is typically across the entire length of SEQ ID NOS: 1 to 74. Such degrees of sequence identity may be, for instance, shown to any one particular sequence selected from SEQ ID NOS: 1 to 74. In one instance, the antisense compound may comprise a sequence with at least 80% sequence identity to any of SEQ ID Nos: 1 to 74 and be from the equivalent region of another human PKB. For instance, whatever PKB the sequence of SEQ ID Nos: 1 to 74 originates from, the sequence with at least 80% sequence identity may be from the other of PKB α, β or γ.

In some instances, the number of nucleotide changes from the sequence of SEQ ID NOS: 1 to 74 may be 3 or less, even more preferably 2 or less and in some instances be only a single nucleotide different. In the case of longer antisense compounds, for instance longer than 24 nucleotides, preferably longer than 26 and more preferably longer than 28 nucleotides, the number of mismatches may, for instance, be 6 or less, preferably 5 or less, more preferably 4 or less and still more preferably any of the number of mismatches mentioned above.

In particularly preferred instances the antisense compounds will consist essentially of the sequence selected from SEQ ID Nos: 1 to 74 and in any especially preferred embodiment the sequence will consist of one of SEQ ID Nos: 1 to 74, particularly of SEQ ID Nos: 1 to 54. The sequence of preferred antisense compounds is indicated in Tables 1 to 9. Table 1 provides examples of preferred PKBα antisense compounds, Table 2 of PKBβ antisense compounds and Table 3 of PKBγ antisense compounds. Table 7 provides additional examples of PKBα antisense compounds, Table 8 additional examples of PKBβ antisense compounds and Table 9 of additional PKBγ antisense compounds.

As indicated in Table 1 the PKBα 925 and 1265 antisense compounds also inhibit PKBβ. As indicated in Table 2 the PKBβ 743 probe also inhibits PKBα. Thus, those antisense compounds, the longer version of such antisense compounds, the equivalent region of non-human mammalian PKBs and/or compounds with one of the levels of sequence identity specified herein may be used to inhibit more than one PKB at once, as indicated. In each case the shorter of the two sequences indicated in the Tables is the preferred sequence, as well as the equivalent region from a non-human mammalian PKB, or a sequence with at least 80% sequence identity to either.

As indicated in Tables 7 to 9 the antisense compounds indicated may be active against more than one PKB form and may be employed accordingly.

Tables 4 and 5 provide some examples of particularly preferred antisense compounds of the invention, again the shorter sequences of each pair is the preferred sequence, as well as the equivalent region from a non-human mammalian PKB, or a sequence with at least 80% sequence identity to either. Table 6 provides a summary of the efficacy of some of the compounds shown in Tables 4 and 5. In one instance, preferred antisense compounds comprise the sequence of SEQ ID Nos: 4, 22 or 44 or sequences with at least 80% sequence identity thereto.

In a particularly preferred instance, the antisense compound comprises the sequence of:

(i) any one of the sequences of SEQ ID Nos. 1 to 4, 19 to 22, and 43 or 44;

(ii) the equivalent region in a non-human mammalian PKB; or

(iii) a sequence with at least 80% sequence identity to the sequence of (i) or (ii).

In a preferred instance, the antisense compound comprises the sequences of any one of SEQ ID Nos 2, 4, 20, 22 and 44, the equivalent region from a non-human mammalian PKB or a sequence with at least 80% sequence identity to the sequence of any of the preceding.

In one instance, the antisense compound comprises the sequence of:
(i) any one of the sequences of SEQ ID Nos: 5 to 18, 23 to 42 and 45 to 54;
(ii) the equivalent region in a non-human mammalian PKB; or
(iii) a sequence with at least 80% sequence identity to any one of the sequences of (i) or (ii).

In a further preferred instance, the antisense compound comprises the sequence of:
(i) any one of the sequences of SEQ ID Nos: 6, 8, 10, 12, 14, 16, 18, 24, 26, 28, 30, 32, 34, 36, 38, 40, 42, 46, 48, 50, 52 and 54;
(ii) the equivalent region in a non-human mammalian PKB; or
(iii) a sequence with at least 80% sequence identity to any one of the sequence of (i) or (ii).

TABLE 1

PKB alpha targets
Target gene: PKBα; Accession no. of gene BC000479; coding sequence 276-1718

| | Target | | |
|---|---|---|---|
| Name | site | Sequence | Advantages |
| | 1651 | AGTGGGGCCTGCGCTCGCTGTCCACACACT | See Table 6 below |
| | | (SEQ ID No. 1) | |
| | 1657 | GCCTGGGCTCGCTGTCCA | See Table 6 below |
| | | (SEQ ID No. 2) | |
| | 1267 | CGTACATGACCACGCCCAGCCCCCACCAGT | See Table 6 below |
| | | (SEQ ID No. 3) | |
| | 1273 | TGACCACGCCCAGCCCCC | See Table 6 below |
| | | (SEQ ID No. 4) | |
| | (684) | GGTCACGCGGTGCTTGGGCTTGGCCAGGGA | |
| | | (SEQ ID No. 5) | |
| HA690AS | 690 | GCGGTGCTTGGGCTTGGC | Tₘ = 77° C., no dimers, no fold back. |
| | | (SEQ ID No. 6) | |
| | (919) | AGCAGAGGCGGTCGTGGGTCTGGAAAGAGT | |
| | | (SEQ ID No. 7) | |
| HA925AS | 925 | GGCGGTCGTGGGTCTGGA | Tₘ = 74° C., no dimers, no fold back, no hairpin. |
| | | (SEQ ID No. 8) | Also targets the same region in PKBβ and hence |
| | | | can be used to knockout both PKB α and β. |
| | (925) | TGACAAAGCAGAGGCGGTCGTGGGTCTGGA | |
| | | (SEQ ID No. 9) | |
| HA931AS | 931 | AGCAGAGGCGGTCGTGGG | Tₘ = 73° C., no dimers, no fold back, no hairpin. |
| | | (SEQ ID No. 10) | |
| | (1112) | TTAATGTGCCCGTCCTTGTCCAGCATGAGG | |
| | | (SEQ ID No. 11) | |
| HA1118A | 1118 | TGCCCGTCCTTGTCCAGC | Tₘ = 72° C., no dimers, no fold back, no hairpin. |
| S | | (SEQ ID No. 12) | |
| | (1259) | ACCACGCCCAGCCCCCACCAGTCCACTGCA | |
| | | (SEQ ID No. 13) | |
| HA1265A | 1265 | CCCAGCCCCCACCAGTCC | Tₘ = 75° C., no dimers, no fold back, no hairpin. |
| S | | (SEQ ID No. 14) | Also targets the same region in PKBβ and hence |
| | | | can be used to knockout both PKB α and β. |
| | (1692) | GCCTCAGGCCGTGCCGCTGGCCGAGTAGGA | |
| | | (SEQ ID No. 15) | |
| HA1698A | 1698 | GGCCGTGCCGCTGGCCGA | Tₘ = 80° C., no hairpin, our studies show this |
| S | | (SEQ ID No. 16) | region to be antisense-active in other PKB |
| | | | isoforms and the SASβ probe of SEQ ID No. 20 |
| | | | binds to the corresponding region in PKB β. |
| | (1712) | GTCCAGCGCAGTCCACCGCCGCCTCAGGCC | |
| | | (SEQ ID No. 17) | |
| HA1718A | 1718 | CGCAGTCCACCGCCGCCT | Tₘ = 79° C., no dimers, no fold back, no hairpin. |
| S | | (SEQ ID No. 18) | |

TABLE 2

PKB beta targets
Target gene: PKBβ; Accession no. of gene M95936; coding sequence 88-1533

| | | | |
|---|---|---|---|
| Name | Target | Sequence | Advantages |

| | | | |
|---|---|---|---|
| | 1507 | TGCTCACTCGCGGATGCTGGCCGAGTAGGA | See Table 6 below |
| | | (SEQ ID No. 19) | |
| | 1513 | CTCGCGGATGCTGGCCGA | See Table 6 below |
| | | (SEQ ID No. 20) | |
| | 1082 | CGTACATGACCACACCCAGCCCCCACCAGT | See Table 6 below |
| | | (SEQ ID No. 21) | |
| | 1088 | TGACCACACCCAGCCCCC | See Table 6 below |
| | | (SEQ ID No. 22) | |
| | (368) | GGATGGCCCGCATCCACTCCTCCCTCTCGT | |
| | | (SEQ ID No. 23) | |
| HB374AS | 374 | CCCGCATCCACTCCTCCC | Tₘ = 73° C., no dimers, no fold back, no hairpin. |
| | | (SEQ ID No. 24) | |
| | (404) | CTGGGGCCCGCTGCTTGAGGCTGTTGGCGA | |
| | | (SEQ ID No. 25) | |
| HB410AS | 410 | CCCGCTGCTTGAGGCTGT | Tₘ = 71° C., no dimers, no fold back, no hairpin. |
| | | (SEQ ID No. 26) | |
| | (422) | AGTCCATGGGGTCCTCGCCTGGGGCCCGCT | |
| | | (SEQ ID No. 27) | |
| HB428AS | 428 | TGGGGTCCTCGCCTGGGG | Tₘ = 78° C., no dimers, no fold back, no hairpin. |
| | | (SEQ ID No. 28) | |
| | (494) | TTTAGCCCGTGCCTTGCTGACCGCCACTTC | |
| | | (SEQ ID No. 29) | |
| HB502AS | 502 | CCGTGCCTTGCTGACCGC | Tₘ = 75° C., no dimers, no fold back, no hairpin. |
| | | (SEQ ID No. 30) | |
| | (737) | AGCACAGGCGGTCGTGGGTCTGGAAGGCAT | |
| | | (SEQ ID No. 31) | |
| HB743AS | 743 | GGCGGTCGTGGGTCTGGA | Tₘ = 75° C., no dimers, no fold back, no hairpin. |
| | | (SEQ ID No. 32) | Also targets the same region in PKBβ and |
| | | | hence can be used to knockout both PKBα and |
| | | | β. |
| | (1070) | CACCCAGCCCCCACCAGTCCACGGCCCGGC | |
| | | (SEQ ID No. 33) | |
| HB1076A | 1076 | GCCCCCACCAGTCCACGG | Tₘ = 76° C., no dimers, no fold back, no hairpin. |
| | | (SEQ ID No. 34) | |
| | (1253) | CGCTGGGCCCCCCACCAAGCCTCTGCTTGG | |
| | | (SEQ ID No. 35) | |
| HB1259AS | 1259 | GCCCCCCACCAAGCCTCT | Tₘ = 74° C., no dimers, no fold back, no hairpin. |
| | | (SEQ ID No. 36) | |
| | (1466) | AGTGGGTCCGCTGGTCCAGCTCCAGTAAGC | |
| | | (SEQ ID No. 37) | |
| HB1472 | 1472 | TCCGCTGGTCCAGCTCCA | Tₘ = 72° C., no hairpin, our studies show this |
| | | (SEQ ID No. 38) | region to be antisense-active in other PKB |
| | | | isoforms and the SASα probe of SEQ No. 2 |
| | | | targets the corresponding region in PKBα. |
| | (1538) | CAGCGAGCGTGCGTCCTCTGCGTGGGCAGA | |
| | | | (SEQ ID No. 39) |
| HB1544AS | 1544 | GCGTGCGTCCTCTGCGTG | Tₘ = 74° C., no dimers, no fold back, no hairpin. |
| | | (SEQ ID No. 40) | |
| | (1553) | ACCCAGCGGTGATGGCAGCGAGCGTGCGTC | |
| | | (SEQ ID No. 41) | |
| HB1559AS | 1559 | CGGTGATGGCAGCGAGCG | Tₘ = 74° C., no dimers, no fold back, no hairpin. |
| | | (SEQ ID No. 42) | |

TABLE 3

PKB gamma targets.
Target gene: PKBγ; Accession no. of gene AF124141; coding sequence 13-1452

| | Target | | |
|---|---|---|---|
| Name | site | Sequence | Advantages |
| | 983 | CCCCTAGGCCCCACCAGTCTAGTGCTCGGC | See Table 6 below |
| | | (SEQ ID No. 43) | |
| | 989 | GGCCCCACCAGTCTACTG | See Table 6 below |
| | | (SEQ ID No. 44) | |
| | (693) | AAATGGAAAAACAGCTCGCCCCCATTAACA | |
| | | (SEQ ID No. 45) | |
| HC699AS | 699 | AAAAACAGCTCGCCCCCA | Tₘ = 71° C., no hairpin. |
| | | (SEQ ID No. 46) | |
| | (995) | CATACATGACAACCCCTAGGCCCCACCAGT | |
| | | (SEQ ID No. 47) | |
| HC1001AS | 1001 | TGACAACCCCTAGGCCCC | Tₘ = 70° C., no hairpin, our studies show |
| | | (SEQ ID No. 48) | this region to be antisense-active in other |
| | | | PKB isoforms and the DASα probe of SEQ |
| | | | ID No. 4 and the DASβ probe of SEQ ID |
| | | | No. 22. |
| | (1385) | AATGCGGCCGCCTCTCATTGTCCATGCAGT | |
| | | (SEQ ID No. 49) | |
| HC1391AS | 1391 | GCCGCCTCTCATTGTCCA | Tₘ = 69° C., no dimers, our studies show |
| | | (SEQ ID No. 50) | this region to be antisense-active in other |
| | | | PKB isoforms and the SASα probe of SEQ |
| | | | ID No. 2 binds to the corresponding region |
| | | | in PKBα. |
| | (1426) | GACTTATTCTCGTCCACTTGCAGAGTAGGA | |
| | | (SEQ ID No. 51) | |
| HC1432AS | 1432 | TTCTCGTCCACTTGCAGA | Tₘ = 61° C., no hairpin, our studies show |
| | | (SEQ ID No. 52) | this region to be antisense-active in other |
| | | | PKB isoforms and the SASβ probe of SEQ |
| | | | ID No. 2 binds to the corresponding region |
| | | | in PKBβ. |
| | (1572) | GTGAAAGGTGGCGAGGGGTGAGGACCCTTG | |
| | | (SEQ ID No. 53) | |
| HC1578AS | 1578 | GGTGGCGAGGGGTGAGGA | Tₘ = 72° C., no dimers, no fold back. |
| | | (SEQ ID No. 54) | |

TABLE 4

| | Target | Accession | | |
|---|---|---|---|---|
| Name | gene | no | Position | Sequence |
| SASα | PKBα | BC000479 | 1657 | GCCTGCGCTCGCTGTCCA |
| | | | | (SEQ ID No. 2) |
| Long SASα | PKBα | BC000479 | 1651 | AGTGGGGCCTGCGCTCGCTGTCCACACACT |
| | | | | (SEQ ID No. 1) |
| SASβ | PKBβ | M95936 | 1513 | CTCGCGGATGCTGGCCGA |
| | | | | (SEQ ID No. 20) |
| Long SASβ | PKBβ | M95936 | 1507 | TGCTCACTCGCGGATGCTGGCCGAGTAGGA |
| | | | | (SEQ ID No. 19) |
| SASγ | PKBγ | AF124141 | 989 | GGCCCCACCAGTCTACTG |
| | | | | (SEQ ID No. 44) |
| Long SASγ | PKBγ | AF12414 | 983 | CCCCTAGGCCCCACCAGTCTACTGCTCGGC |
| | | | | (SEQ ID No. 43) |
| DASα | PKBα | BC000479 | 1273 | TGACCACGCCCAGCCCCC |
| | | | | (SEQ ID No. 4) |
| Long DASα | PKBα | BC000479 | 1267 | CGTACATGACCACGCCCAGCCCCCACCAGT |
| | | | | (SEQ ID No. 3) |
| DASβ | PKBβ | M95936 | 1088 | TGACCACACCCAGCCCCC |
| | | | | (SEQ ID No. 22) |
| Long DASβ | PKBβ | M95936 | 1082 | CGTACATGACCACACCCAGCCCCCACCAGT |
| | | | | (SEQ ID No. 21) |

TABLE 5
Antisense probes and control mismatch oligonucleotides

| | | |
|---|---|---|
| Single antisense probes | | |
| SASα - Single Antisense α - | GCCTGCGCTCGCTGTCCA | (SEQ ID No. 2) |
| SASβ - Single Antisense β - | CTCGCGGATGCTGGGCGA | (SEQ ID No. 20) |
| SASγ - Single Antisense γ - | GGCCCCACCAGTCTACTG | (SEQ ID No. 44) |

Single mismatch control probes

| | | |
|---|---|---|
| Single MM(α) | GACTCCTCTCAATGTTCA | (SEQ ID No. 75) |
| Single MM(β) | CTATTGGATGATGGATGA | (SEQ ID No. 76) |
| Single MM(γ) | AGCACCAACAATCAACGG | (SEQ ID No. 77) |

Double antisense probes against PKBα and β - DAS(αβ)

| | | |
|---|---|---|
| DASα - Double antisense α - | TGACCACGCCCAGCCCCC | (SEQ ID No. 4) |
| DASβ - Double antisense β - | TGAGCACACCCAGCCCCC | (SEQ ID No. 22) |

Double mismatch control probes - Double MM(αβ)

| | | |
|---|---|---|
| MMα | TGAACAAGCACAGCACCC | (SEQ ID No. 78) |
| MMβ | TGAACAAACACAGCACCC | (SEQ ID No. 79) |

TABLE 6

| | | % Inhibition of | % Inhibition of | % Inhibition of |
|---|---|---|---|---|
| Title of Antisense treatment | Oligonucleotide(s) | PKBα protein | PKBβ protein | PKBγ protein |
| Single AS(α) | SASα | 91 | ns | nd |
| Single AS(β) | SASβ | ns | 92 | nd |
| Single AS(γ) | SASγ | ns | ns | 96 |
| Double AS(α, β) | DASα + DASβ | 94 | 98 | ns |
| Triple AS(α, β, γ) | DASα + DASβ + | 93 | 94 | 94 |
| | SASγ | | | |

TABLE 7

Additional PKB alpha targets
Target gene: PKBα; Accession no. of gene BC000479; coding sequence 276-1718

| | | | |
|---|---|---|---|
| Name | Target | Sequence | Advantages |

| | | | |
|---|---|---|---|
| | (556) | GGATGGCGGTTGTCCACTCCTCCCGCTCCT | |
| | | (SEQ ID No. 55) | |
| HAS62AS | 562 | CGGTTGTCCACTCCTCCC | our studies show this region to be antisense- |
| | | (SEQ ID No. 56) | active in other PKB isoforms and HB374AS |
| | | | probe of SEQ No. 24 targets the corresponding |
| | | | region in PKBβ. |
| | (592) | CCTCCTCCTGCTTCTTGAGGCCGTCAGCCA | |
| | | (SEQ ID No. 57) | |
| HAS98AS | 598 | CCTGCTTCTTGAGGCCGT | our studies show this region to be antisense- |
| | | (SEQ ID No. 58) | active in other PKB isofornis and HB410AS |
| | | | probe of SEQ No. 26 targets the corresponding |
| | | | region in PKBβ. |
| | (962) | AGGTGGAAGAACAGCTCGCCCCCGTTGGCG | |
| | | (SEQ ID No. 59) | |
| HA968AS | 968 | AAGAACAGCTCGCCCCCG | our studies show this region to be antisense- |
| | | (SEQ ID No. 60) | active in other PKB isoforms and HC699AS |
| | | | probe of SEQ No. 46 targets the corresponding |
| | | | region in PKBγ. |
| | (1438) | CCTCGGAGCCCCCGCCAAGCCTGTGCTTGG | |
| | | (SEQ ID No. 61) | |
| HA1444A | 1444 | AGCCCCCGCCAAGCCTCT | our studies show this region to be antisense- |
| S | | (SEQ ID No. 62) | active in other PKB isoforms and HB1259AS |
| | | | probe of SEQ No. 36 targets the corresponding |
| | | region in PKBβ. | |

TABLE 8

Additional PKB beta targets
Target gene: PKBβ; Accession no. of gene M95936; coding sequence 88-1533

| | | | |
|---|---|---|---|
| Name | Target | Sequence | Advantages |

| | | | |
|---|---|---|---|
| | (780) | AGGTGGAAGAACAGCTCACCCCCGTTGGCA | |
| | | (SEQ ID No 63) | |
| HB786AS | 786 | AAGAACAGCTCACCCCCG | our studies show this region to be antisense-active in |
| | | (SEQ ID No 64) | other PKB isoforms and HC699AS probe of SEQ No. |
| | | | 46 targets the conesponding region in PKBγ. |
| | (927) | TTGATGTGGCCATCTTTGTCCAGCATGAGG | |
| | | (SEQ ID No 65) | |
| HB933AS | 933 | TGGCCATCTTTGTCCAGC | our studies show this region to be antisense-active in |
| | | (SEQ ID No. 66) | other PKB isoforms and HA1118AS probe of SEQ |
| | | | No. 12 targets the corresponding region in PKBα. |

TABLE 9

Additional PKB gamma targets.
Target gene: PKBγ; Accession no. of gene AF124141; coding sequence 13-1452

| | | | |
|---|---|---|---|
| Name | Target | Sequence | Advantages |

| | | | |
|---|---|---|---|
| | (290) | GGATAGCTTCTGTCCATTCTTCCCTTTCCT | |
| | | (SEQ ID 67) | |
| HC296AS | 296 | CTTCTGTCCATTCTTCCC | our studies show this region to be antisense-active in |
| | | (SEQ ID No 68) | other PKB isoforms and HB374AS probe of SEQ No. |
| | | | 24 targets the correspondin region in PKBβ. |
| | (326) | CCTCTTCTTGCCTCTGCAGTCTGTCTGCTA | |
| | | (SEQ ID No 69) | |
| HC332AS | 332 | CTTGCCTCTGCAGTCTGT | our studies show this region to be antisense-active in |
| | | (SEQ ID No 70) | other PKB isoforms and HB410AS probe of SEQ No. |
| | | | 26 targets the corresponding region in PKBβ |
| | (840) | TTTATGTGGCCATCTTTGTCCAGCATTAGA | |
| | | (SEQ ID No 71) | |
| HC846AS | 846 | TGGCCATCTTTGTCCAGC | our studies show this region to be antisense-active in |
| | | (SEQ ID No. 72) | other PKB isoforms and HA1118AS probe of SEQ |
| | | | No. 12 targets the corresponding region in PKBα |
| | (1166) | CATCTGGTCCTCCACCAAGGCGTTTATTTG | |
| | | (SEQ ID NO 73) | |
| HC1172AS | 1172 | GTCCTCCACCAAGGCGTT | our studies show this region to be antisense-active in |
| | | (SEQ ID NO 74) | other PKB isoforms and HB1259AS probe of SEQ |
| | | | No. 36 targets the corresponding region in PKBβ. |

In one embodiment, where it is desired to inhibit PKBα, the antisense compound comprises a sequence selected from any one of SEQ ID Nos: 1 to 18 and 55 to 62 and in particular any one of SEQ ID Nos: 1 to 18, the equivalent sequence from a non-human mammalian PKB or a sequence with at least 80% sequence identity to any of the preceding. In one instance, where it is only desired to inhibit PKBα, or at least it is intended not to inhibit PKBβ, the compound may comprise the sequence of any one of SEQ ID Nos 1 to 6, 9, 10, 11, 12, and 15 to 18, the equivalent sequence from a non-human mammalian PKB or a sequence with at least 80% sequence identity to any of the preceding. In all of the preceding, the shorter sequence of each pair indicated is the preferred sequence as are the equivalent sequence from a non-human mammalian PKB or a sequence with at least 80% sequence identity to either. In a particularly, preferred instance, the antisense compound comprises the sequence of any one of SEQ ID Nos 1 to 4 and in particular SEQ ID Nos 2 or 4.

In a further particularly preferred instance, the antisense compound may comprise the sequence of SEQ ID Nos: 14, 12, 18 or 4. The antisense compound may comprise the equivalent sequence from a non-human mammalian PKB or a sequence with at least 80% sequence identity to any of the preceding sequences.

In a further preferred instance the antisense compound may comprise the sequence of any one of SEQ ID Nos: 6 to 18, the equivalent sequences from a non-human mammalian PKB or a sequence with at least 80% sequence identity to any of the preceding. Preferably the shorter sequence of each pair is employed. In one preferred instance, the order of descending preference is that the antisense compound comprises the sequences of SEQ ID Nos: 14, 12, 18, 16, 10, 6 or 8. The equivalent sequence from a non-human mammalian PKB or a sequence with 80% sequence identity may be employed. In one instance, the sequence may be selected from the top five, four, three or two most preferred in the order of preference indicated. In a particularly, preferred embodiment the sequence may comprises that of SEQ ID NO: 14, 12, or 18, preferably SEQ ID NO: 14 or 12 and in particular SEQ ID NO: 14. Such compounds may in particular be used to inhibit PKB-α.

In one embodiment, where it is desired to inhibit PKBβ, the antisense compound comprises a sequence selected from any one of SEQ ID Nos: 19 to 42 and 63 to 66 and in particular any one of SEQ ID Nos: 19 to 42, the equivalent sequence from a non-human mammalian PKB or a sequence with at least 80% sequence identity to any of the preceding. In one instance, where it is only desired to inhibit PKBβ, or at least it is intended not to inhibit PKBα, the compound may comprise the sequence of any one of SEQ ID Nos 19 to 30 and 33 to 42, the equivalent sequence from a non-human mammalian PKB or a sequence with at least 80% sequence identity to any of the preceding. In all of the preceding, the shorter sequence of each pair indicated is the preferred sequence as are the equivalent sequence from a non-human mammalian PKB or a sequence with at least 80% sequence identity to either. In a particularly, preferred instance, the antisense compound comprises the sequence of any one of SEQ ID Nos 19 to 22 and in particular 20 and 22.

In one particularly preferred instance, the antisense compound may comprise the sequence of any one of SEQ ID Nos: 38, 24, 36, 26, 40, 30 and 20. The equivalent sequence from a non-human mammalian PKB may be employed as may sequences with at least 80% sequence identity to any of the preceding.

In a further preferred instance, the antisense compound may comprise the sequence of any one of SEQ ID Nos 23 to 42, the equivalent sequence from non-human mammalian PKB or a sequence with at least 80% sequence identity to any of the preceding. Preferably, the shorter sequence of each pair is employed. In one preferred instance, in descending preference, the antisense compound comprises the sequence of SEQ ID NO: 38, 34, 36, 26, 40, 30, 34, 24, 28 or 42. In one instance, the sequence may be selected from the top seven, six, five, four, three or two of the most preferred of those sequences in the order of preference. In a particularly preferred embodiment the sequence may comprise that of SEQ ID NO: 38, 34 or 36, preferably SEQ ID NO: 38 or 34 and even more preferably SEQ ID NO: 38. Such compounds may in particular be used to inhibit PKB-β.

In one embodiment, where it is desired to inhibit PKBγ, the antisense compound comprises a sequence selected from any one of SEQ ID Nos: 43 to 54 and 67 to 74 and in particular any one of SEQ ID Nos: 43 to 54, the equivalent sequence from a non-human mammalian PKB or a sequence with at least 80% sequence identity to any of the preceding. In all of the preceding, the shorter sequence of each pair indicated is the preferred sequence as are the equivalent sequence from a non-human mammalian PKB or a sequence with at least 80% sequence identity to either. In a particularly, preferred instance, the antisense compound comprises the sequence of any one of SEQ ID Nos 43 and 44 and in particular SEQ ID No. 44.

In one instance, in descending preference, the antisense compound may comprise the sequence of SEQ ID Nos: 50, 46, 54, 52 or 48. In one instance, the sequence may be selected from the top four, three or two most preferred sequences from the order of preference. In a preferred instance, the compound will comprise the sequence of SEQ ID Nos: 50 or 46 and in particular SEQ ID No. 50.

In one particularly preferred instance, the antisense compound may comprise the sequence of SEQ ID No: 50, 46 or 44. The equivalent sequence from a non-human mammalian PKB may be employed as may sequences with at least 80% sequence identity.

The sequence of the shorter of the pairs of sequence indicated above, the equivalent sequence from a non-human mammalian PKB or a sequence with at least 80% sequence identity to either is preferred. Thus, Tables 1 to 8 indicate pairs of long and short sequences and antisense sequences comprising the shorter of each pair are preferred, as well as the equivalent region from a non-human mammalian PKB or a sequence with at least 80% sequence identity to either. The sequence of specific antisense compounds indicated is particularly preferred. In one instance, where groups of preferred sequences are indicated herein, the invention also provides a further group which includes the equivalent longer sequences as well. In another instance, just the shorter antisense compounds may be employed.

In one particularly preferred instance the antisense compounds consist of the sequences of SEQ ID Nos: 4, 22, or 44, the equivalent region from a non-human mammalian PKB or a sequence with 80% sequence to any of the preceding. Antisense compounds consisting of such sequences are preferred. In one embodiment antisense compounds comprising the sequence of SEQ ID Nos 4 or 22, the equivalent region from a non-human mammalian PKB or a sequence with at least 80% sequence identity to any of the preceding are provided.

The antisense compounds of the invention are capable of inhibiting wholly or partially PKB α, β and/or γ. The level of inhibition of individual forms of PKB may preferably be at least 25%, more preferably at least 50%, even more preferably at least 60%, still more preferably at least 70%, and even more preferably at least 80%. In some instances, the level of inhibition may be at least 75%, preferably at least 85% and even more preferably at least 90%. In some instances the level of inhibition may be at least 95%. Combinations of antisense compounds of the invention may be employed to inhibit wholly or partially several PKB forms at once. Thus, two, or all of, PKB α β and γ may be inhibited and may, for instance, be inhibited to any of the levels mentioned herein either individually or collectively. In preferred instances, the invention may be used to inhibit PKB α and β, PKB α and γ, or PKB β and γ and in a further preferred embodiment PKB α, β and γ may be wholly or partially inhibited. In particular, kinase activity may be inhibited.

In one instance, a PKB gene, or part of a PKB gene, of an individual to be treated may be sequenced to design the antisense compound on the equivalent regions indicated herein.

In one instance, the region from another PKB corresponding to the region bound by one of the antisense compounds indicated herein may be employed. For instance, as indicated in Tables 1 to 3 and 7 to 9 above some of the specific antisense compounds bind to the equivalent region in different PKB isoforms. Sequences from equivalent non-human PKBs and sequences with 80% sequence identity to either may also be employed. An equivalent region may be identified, for instance, by aligning the two sequences and picking the region of appropriate length.

Antisense compounds of the invention are capable of wholly or partially inhibiting PKBα, β and/or γ. Any of the methods described herein, and in particular the methods used in the Examples, may be employed to assess such inhibition. Measurement of other parameters indicated herein as influenced by PKB activity may also be used to measure PKB inhibition. The level of pSER 473-PKB may, for instance, be measured in one instance.

The antisense compounds are preferably antisense oligonucleotides. In an especially preferred instance the compounds are phosphorothioate antisense compounds. Instances of other preferred antisense compounds include oligonucleotides containing modified backbones or non-natural internucleoside linkages. In a preferred instance at least one modified internucleoside linkage may be present. In some instances at least two, preferably at least three and more preferably at least four such linkages may be present. In other instances, such linkages may be absent.

Antisense compounds having modified backbones may encompass those that retain a phosphorus atom in the backbone and those that do not have a phosphorus atom in the backbone. Modified oligonucleotides that do not have a phosphorus atom in their internucleoside backbone can also be considered to be oligonucleosides.

In a preferred instance modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

In other instances, both the sugar and the internucleoside linkage, i.e., the backbone, of the nucleotide units are replaced with other groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

Particularly preferred instances are oligonucleotides with phosphorothioate backbones and oligonucleosides with heteroatom backbones, and in particular —CH2-NH—O—CH2-, —CH2-N(CH3)-O—CH2— [known as a methylene (methylimino) or MMI backbone], —CH2-O—N(CH3)-CH2-, —CH2-N(CH3)-N(CH3)-CH2- and —O—N(CH3)-CH2-CH2- [wherein the native phosphodiester backbone is represented as —O—P—O—CH2-]. Also preferred are oligonucleotides having morpholino backbone structures.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly preferred are O[(CH2)nO]m CH3, O(CH2)nOCH3, O(CH2)nNH2, O(CH2)nCH3, O(CH2)nONH2, and O(CH2)nON[(CH2)nCH3)]2, where n and m are from 1 to about 10. Other preferred oligonucleotides comprise one of the following at the 2′ position: C1 to C10 lower allyl, substituted lower alkyl, alkaryl, aralkyl, O-alkaryl or O-aralkyl, SH, SCH3, OCN, Cl, Br, CN, CF3, OCF3, SOCH3, SO2 CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Possible modifications includes 2′-methoxyethoxy (2′-O—CH2CH2OCH3, also known as 2′-O-(2-methoxyethyl) or 2′-MOE) i.e., an alkoxyalkoxy group. A further possible modification includes 2′-dimethylaminooxyethoxy, i.e., a O(CH2)2ON(CH3)2 group, also known as 2′-DMAOE. In one preferred instance the antisense compound of the invention does not have a 2′ MOE or 2′-DMAOE modification.

Other possible modifications include 2′-methoxy (2′-O—CH3), 2′-aminopropoxy (2′-OCH2CH2CH2NH2) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Antisense compounds may also include base modifications or substitutions. As used herein, “unmodified” or “natural” bases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Instances of bases which may be used include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions may increase nucleic acid duplex stability by 0.6-1.2° C.

Another modification of the antisense compounds of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Bebmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. In some instances of the invention the antisense compound may not be so modified.

It is not necessary for all positions in a given compound to be uniformly modified, and more than one of the modifications discussed here may be incorporated in a single compound or even at a single nucleoside within an oligonucleotide.

The antisense compounds may for instance be made through the well-known technique of solid phase synthesis. Any other means for such synthesis may be employed. It is well known to use similar techniques to prepare antisense compounds and in particular oligonucleotides, such as the phosphorothioates and alkylated derivatives.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the invention also encompasses prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the antisense compounds of the invention may be prepared as SATE [(S-acetyl-2-thioethyl) phosphate] derivatives.

### Combinations

The present invention provides combinations of at least two different antisense compounds collectively capable of inhibiting wholly or partially at least two of PKB α, β and γ. In one preferred instance such a combination is collectively capable of inhibiting wholly or partially all of PKB α, β and γ.

The invention also provides a combination comprising an antisense compound of the invention and at least one other antisense compound. In a preferred instance the other antisense compound is one which wholly or partially inhibits PKB α, β and/or γ. The sequences of PKB α, β and γ are publicly available and may be used to generate the said other antisense compounds. In particular, the sequences of the PKBα gene is provided as Accession Number BC000479, the sequence of the PKBα gene as Accession Number M95936 and the sequence of the PKBα genes as AF124141.

In an especially preferred embodiment the present invention also provides combinations comprising at least two antisense compounds of the invention.

In a further preferred instance the present invention provides such a combination which comprises:
(i) a combination of antisense compounds collectively capable of inhibiting PKB α and β wholly or partially;
(ii) a combination of antisense compounds collectively capable of inhibiting PKB α and γ wholly or partially;
(iii) a combination of antisense compounds collectively capable of inhibiting PKB β and γ wholly or partially; and/or
(iv) a combination of antisense compounds collectively capable of inhibiting PKB α, β and γ wholly or partially

In a particularly preferred instance such combinations may comprise at least two antisense compounds collectively capable of inhibiting PKB α, β, and/or γ wholly or partially. Such treble combinations can be used to eliminate wholly or partially all forms of PKB activity. In other instances, it may be preferable to employ either single antisense compounds or combinations to inhibit particular PKBs. Such combinations and antisense compounds may be used to selectively inhibit PKBs and hence tailor treatment to particular disease conditions where one, or two of the PKBs, but not all are implicated including any such conditions mentioned herein. In some cases particular PKB antisense compounds may inhibit more than one PKB and that may be taken into consideration when designing combinations.

In one instance, a combination may comprise at least two antisense compounds discussed herein. In particular, a combination may comprise at least two different antisense compounds selected from Tables 1 to 3 and 7 to 9, with at least two of the antisense compounds being chosen from different Tables. Antisense compounds comprising the equivalent region from a non-human mammalian PKB or comprising a sequence with at least 80% sequence identity to the preceding may also be employed. Combinations to inhibit at least two, or all of, PKBα, β and γ may be provided by selecting appropriate inhibitors of PKB α, β and γ indicated in Tables 1, 2, 3 and 7 to 9. In a preferred instance a combination may comprise at least one, preferably at least two and in some instances at least three of the antisense compounds indicated in Table 5. Equivalent regions from nonhuman mammalian PKBs or sequences with at least 80% sequences identity may be employed. The shorter of each sequence pair indicated is the preferred sequence.

In a preferred instance of the invention a combination is provided comprising two or more antisense compounds of the invention where:
at least one antisense compound comprises the sequences of SEQ ID No: 4 or a sequence with 80% sequence identity thereto; and
at least at least one antisense compound comprises the sequences of SEQ ID No: 22 or a sequence with 80% sequence identity thereto.

In one instance such a combination also comprises at least one antisense compound comprising the sequence of SEQ ID No: 44 or a sequence with at least 80% sequence identity thereto.

In one instance, the combination may include an antisense compound comprising the sequence of any one of, in descending preference, SEQ ID Nos: 6, 12, 18, 16, 10, 6 and 8. In another it may comprise an antisense compound comprising the sequence of any one of, in descending order of preference, SEQ ID Nos: 38, 24, 36, 26, 40, 30, 34, 24, 28 and 42. In a further preferred instance, the combination may include an antisense compound comprising the sequence of any one of, in descending preference, SEQ ID Nos: 50, 46, 54, 52 and 48. The sequence may be selected from the top six, five, four, three or two most preferred in the above indicated orders of preference in some instances.

Any of the combinations mentioned in the Examples are provided as are combinations based on such sequences, such as, for instance sequences with at least 80% sequence identity thereto or have any of the variations described herein. Combinations of antisense compounds from particular antisense compound groupings indicated herein may be employed

The combinations of the invention may, in some instances comprise, two and preferably three antisense compounds. In others they may comprises at least two, preferably at least three, more preferably at least four, still more preferably at least five antisense compounds. Double or triple combinations are especially preferred.

The present invention also provides a combination comprising:

(i) an antisense compound or combination of the invention; and

(ii) a therapeutic agent other than (i).

In particular, the therapeutic agent (ii) may be an anticancer agent, an agent for treating diabetes, an agent for treating a degenerative neurological disorder and/or an agent for treating a mental disorder. Particularly preferred instances of degenerative neurological conditions include Huntington's disease and Alzheimer's. A preferred mental disorder is bipolar disorder and schizophrenia.

Examples of possible therapeutic agents which may be used in combination with an antisense compound of the invention include tamoxifen, doxorubicin, cisplatin, paclitaxel and/or docetaxel. The use of combinations comprising one or more of the antisense compounds of the invention in combination with other therapeutic agents is a preferred instance as such an approach may raise the efficacy of existing treatments for disorders. The compounds may be administered to a subject undergoing radiotherapy and/or following or preceding surgery to remove a tumour.

The present invention also provides for a polynucleotide capable of expressing an antisense compound of the invention or a combination of the invention. Though in many instances the antisense compounds of the invention are synthetically generated, expression from such a polynucleotide may be employed both to generate the antisense compound for harvest and subsequent therapeutic use and also for in vivo use. Methods for expressing antisense compounds are well known in the art and may be employed. In a particularly preferred instance the polynucleotide for expression may be a vector.

### Medicaments and Treatments

The compounds of the invention may be used to treat a variety of disorders. Thus, the invention provides for an antisense compound, a combination, a polynucleotide or a host cell of the invention for use in a method of treatment of the human or animal body by therapy. In particular, disorders which may be treated include cancer, diabetes, degenerative neurological disorders and mental disorders. Neurological disorders include Alzheimer's and Huntington's disease. Examples of mental disorders include bipolar disorder and schizophrenia.

In a particularly preferred instance, the invention may be used to treat cancers. Particularly preferred cancers to be treated include ovarian, cervical, breast, pancreatic, prostate, stomach, lung, colorectal, glioblastoma, prostate and endometrial cancers. In one preferred instance the cancer to be treated is a solid tumour. In a particularly preferred instance, the cancer is a breast cancer. In another preferred instance it is a prostate cancer. In a further preferred instance it is a neuroblastoma.

Examples of cancers that may be treated according to the invention include primary and secondary cancers. The cancer may be, for example, a leukaemia, a lymphoma, a sarcoma, a carcinoma, or an adenocarcinoma. Specific types of cancer that may be treated according to the invention include breast, colon, brain, lung, ovarian, pancreatic, stomach, skin, testicular, head, neck and tongue cancers. Cancers include breast cancers, B and T cell leukaemias and lymphomas, head and neck cancers. In one instance, the cancer to be treated may show resistance to a particular treatment other than that of the invention.

The invention provides for the use of an antisense compound, a combination, a polynucleotide or a host cell of the invention in the manufacture of a medicament for treating cancer, diabetes, a degenerative neurological condition and/or a mental disorder. In particular, the antisense compounds and combinations of the invention may be used for such a purpose.

The invention also provides products comprising:

(i) an antisense compound or a combination of the invention; and

(ii) a therapeutic agent other than (i)

for the simultaneous, separate or sequential use in the treatment of cancer, diabetes, a degenerative neurological condition and/or a mental disorder.

The invention also provides for a method of treating cancer, diabetes, a degenerative neurological condition and/or a mental disorder which comprises administering an effective amount of an antisense compound, a combination, a polynucleotide or a host cell of the invention to a subject with such a disorder.

The invention may be used to inhibit one, two or all of the PKBs including any of the combinations mentioned herein and to any of the levels mentioned herein. In one embodiment the invention may be used to treated a disorder associated with alteration in one or two, but not all of the PKBs. Thus, the invention may be used to treat subjects by selectively inhibiting particular PKBs.

For instance, the individual PKB isoforms, PKBα, PKBβ and PKBγ, may have unique as well as common functions within the cell. Relative to PKBα and PKBγ, PKBβ plays the predominant role in phosphorylating and inactivating GSK-3 in response to insulin. PKBβ may also be the major isoform that signals increased insulin-stimulated glucose transport. Individual isoforms of PKB are also selectively elevated in many cancer types. In particular, PKBα activity is increased in prostate or breast cancers, while PKBβ amplification occurs in pancreatic or ovarian cancers. PKBγ is elevated in many melanomas. Antisense compounds specific to each PKB may be used to treat such conditions/modulate such parameters.

The subject to be treated using the invention is typically mammalian. In an especially preferred embodiment the subject to be treated is human. The subject may be a domestic animal or an agriculturally important animal. The animal may, for example, be a sheep, pig, cow, bull, poultry bird or other commercially farmed animal. The animal may be a domestic pet such as a dog, cat, bird, or rodent. The rodent may be a mouse or rat. In a preferred embodiment the animal may be a cat or other feline animal. The animal may be a monkey such as a non-human primate. For example, the primate may be a chimpanzee, gorilla, or orangutan.

### Formulations and Administration

The anti-sense polynucleotides may, for instance, be formulated for parenteral, intramuscular, intracerebral, intravenous, subcutaneous or transdermal administration. In one particularly preferred instance, intravenous administration may be employed and, for instance, continuous intravenous infusion may be employed.

The antisense polynucleotides may also preferably administered topically (at the site to be treated). In some instances the route of delivery may be pulmonary (for instance via inhalation of powders or aerosols, including by nebulizer; intratracheal, intranasal, epidermal and transdermal administration), oral or parenteral. Parenteral routes of administration includes intravenous, intraarterial, subcutaneous, intraperitoneal or intramuscular injection or infusion. The route of delivery may be intracranial. Intrathecal or intraventricular administration may be employed. In a particularly preferred instance the route of administration may be topical. In one instance, an antisense compound or polynucleotide of the invention is delivered via biolistic delivery using needleless injection, particularly via carrier particles coated or comprising the compound/polynucleotide, preferably via coated gold particles.

Suitably the antisense polynucleotides are combined with a pharmaceutically acceptable carrier, vehicle or diluent to provide a pharmaceutical composition. Suitable pharmaceutically acceptable carriers or vehicles include any of those commonly used for the routes of administration mentioned herein and in particular for topical administration. The topical formulation may be in the form of a cream, ointment, gel, emulsion, lotion or paint. The formulation of the invention may also be presented in the form of an impregnated dressing. Conventional pharmaceutical carriers, aqueous, powder or oily bases, thickeners and the like may be necessary or desirable.

The compositions may, for instance, be in the form of suppositories, sprays, liquids and powders, transdermal patches, ointments, lotions, creams, gels, drops. Compositions may include, particularly for oral administration, powders or granules, suspensions or solutions in water or non-aqueous media, capsules, sachets or tablets. Pharmaceutical compositions of the present invention include solutions, emulsions, and liposome-containing formulations. In one preferred instance of the invention the antisense compounds of the invention and the various other moieties of the invention may be delivered via liposomes. Thus, the invention provides liposomes comprising the antisense compounds, combinations and/or polynucleotides of the invention. Methods of preparing liposomes are well know in the art and may be employed.

The compositions of the present invention may be formulated as tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilisers. In some instances, the compositions of the invention may be formulated as foams. Pharmaceutical foams include emulsions, microemulsions, creams, jellies and liposomes. The compositions of the invention may be formulated in dosage form.

Possible carrier materials, particularly for topical administration, include any carrier or vehicle commonly used as a base for creams, lotions, gels, emulsions, lotions or paints for topical administration. Examples include emulsifying agents, inert carriers including hydrocarbon bases, emulsifying bases, non-toxic solvents or water-soluble bases. Suitable examples include lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

The pharmaceutical carrier or diluent employed may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphate; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes. The pharmaceutical preparation may be formulated for intravenous administration or indeed for any of the routes indicated herein.

Liquid dispersions for oral administration may be syrups, emulsions and suspensions. The syrups may contain as carriers, for example, saccharose or saccharose with glycerine and/or mannitol and/or sorbitol. Suspensions and emulsions may contain as carrier, for example a natural gum, agar, sodium alginte, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

In particular instances the pharmaceutically acceptable carrier or vehicle is a gel, suitably a nonionic polyoxyethylene-polyoxypropylene copolymer gel, for example, a Pluronic gel, preferably Pluronic F-127 (BASF Corp.). This gel is particularly preferred as it is a liquid at low temperatures but rapidly sets at physiological temperatures, which confines the release of the ODN component to the site of application or immediately adjacent that site.

An auxiliary agent such as casein, gelatin, albumin, glue, sodium alginate, carboxymethylcellulose, methylcellulose, hydroxyethylcellulose or polyvinyl alcohol may also be included in the formulations of the invention. The pharmaceutical composition may be formulated to provide sustained release of the compounds and other moieties of the invention.

Possible formulations may include a surfactant to assist with oligodeoxynucleotide cell penetration or the formulation may contain any suitable loading agent. Any suitable non-toxic surfactant may be included, such as DMSO. Alternatively a transdermal penetration agents such as urea may be included. Uptake of nucleic acids by mammalian cells is enhanced by several known transfection techniques for example through the use of transfection agents. The formulation which is administered may contain such agents. Example of these agents include cationic agents (for example calcium phosphate and DEAE-dextran) and lipofectants (for instance lipofectam and transfectam). Other possible surfactants include non-ionic, anionic and cationic surfactants. Examples of surfactants that may be used include, for example, polyoxyethylene derivatives of fatty acid partial esters of sorbitol anhydrides, such as for example, Tween 80, Polyoxyl 40 Stearate, Polyoxy ethylene 50 Stearate, fusieates, bile salts and Octoxynol.

Typically, antisense compounds will be administered to the subject to be treated. However, in some instance a polynucleotide, vector or cell capable of expressing the antisense compounds may be administered or used to produce antisense compounds for administration.

The antisense compound may therefore be expressed in a cell from a suitable vector. A suitable vector is typically a recombinant replicable vector comprising a sequence which, when transcribed, gives rise to the polynucleotide (typically an RNA). Typically, the sequence encoding the polynucleotide is operably linked to a control sequence which is capable of providing for the transcription of the sequence giving rise to the polynucleotide. The term “operably linked” refers to a juxtaposition wherein the components described are in a relationship permitting them to function in their intended manner. A control sequence “operably linked” to a sequence giving rise to an antisense RNA is ligated in such a way that transcription of the sequence is achieved under conditions compatible with the control sequences. Preferred cells are mammalian cells, in particular human cells.

The vectors may be for example, plasmid or virus vectors provided with an origin of replication, optionally a promoter for transcription to occur and optionally a regulator of the promoter. The vectors may contain one or more selectable marker genes, for example an ampicillin resistance gene in the case of bacterial plasmid or a neomycin resistance gene for a mammalian vector. Vectors may be used in vitro, for example for the production of antisense RNA, or used to transfect or transform a host cell. The vector may also be adapted for used in vivo, for example in a method of gene therapy.

Promoters/enhancers and other expression regulation signals may be selected to be compatible with the host cell for which the expression vector is designed. For example, mammalian promoters, such as beta-actin promoters, may be used. Viral promoters may also be used, for example the Moloney murine leukaemia virus long terminal repeat (MMLV LTR), the promoter rous sarcoma virus (RSV) LTR promoter, the SV40 promoter, the human cytomegalovirus (CMV) IE promoter, herpes simplex virus promoters or adenovirus promoters. All these promoters are readily available in the art. Preferred promoters are tissue specific promoters, for example promoters driving expression specifically within tissue effected by a particular disorder. Preferred promoters can give rise to expression in mammalian cells.

Vectors may further include additional sequences, flanking the sequence giving rise to the antisense polynucleotide, which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination.

Examples of suitable viral vectors include retroviruses, including lentiviruses, adenoviruses, adeno-associated viruses and herpes simplex viruses. Gene transfer techniques using such viruses are will known to those skilled in the art. Retrovirus vectors, for example, may be used to stably integrate the polynucleotide giving rise to the antisense compound into the host genome. Replication-defective adenovirus vectors by contrast remain episomal and therefore allow transient expression.

A suitable therapeutic agent of the invention is administered to a patient. The dose of a suitable agent may be determined according to various parameters, especially according to the substance used; the age, weight and condition of the patient to be treated; the route of administration; and the required regimen. Again, a physician will be able to determine the required route of administration and dosage for any particular patient. For instance, in some cases a dosage range of from 0.1 to 20 mg/kg/day, preferably from 0.5 to 15 mg/kg/day and even more preferably from 1 to 10 mg/kg/day may be administered. In some instances, a dose of from 1 to 8, and preferably from 2 to 7 mg/kg/day may be employed. In some instances, a dosage of from 4 to 8 and preferably from 5 to 7 mg/kg/day may be employed. In other instances, a dosage of from 0.5 to 4, preferably from 1 to 3 and even more preferably from 1 to 2 mg/kg/day may be employed. The concentration of antisense compound achieved may, in some instances be from 1 to 20 μM, preferably from 2 to 10 μM, more preferably from 3 to 8 μM and still more preferably from 4 to 7 μM. Such concentrations may be that achieved at the effected site.

The antisense compounds will be administered for an appropriate period of time. In some instances, the compounds may be administered from 1 to 50 days and preferably from 5 to 25 days. In one instance the compound may be administered from 1 to 15, preferably from 3 to 10 and even more preferably from 4 to 7 days and in particular 6 days. In other instances, the compound may be administered for from 5 to 30, preferably from 10 to 25, more preferably from 15 to 23 days and in particular for 21 days. In a particularly preferred instance, the compounds are administered continuously and in particular by continuous intravenous infusion. Examples of regimens which may be employed are provided in Tolcher et al (2004) Clinical Cancer research, Vol. 10: 5048-5057 and Advani et al (2004) Cancer 100: 321-326.

Appropriate dosages may depend on a variety of factors, for example, body weight, according to the activity of the specific agent the age, weight and conditions of the subject to be treated, the type and severity of the degeneration and the frequency and route of administration. Such a dose may be given, for example, once only, or more than once for example 2, 3, 4 or 5 times. The dose may be given, for example daily, every other day, weekly or monthly.

The routes of administration and dosages described above are intended only as a guide since a skilled physician will be able to determine readily the optimum route of administration and dosage for any particular patient and condition.

The therapeutic agent may be administered by direct injection into the site to be treated. For instance a composition of the invention may be injected into a tumour or in a vessel so it is delivered to a tumour. Preferably, the agent is combined with a pharmaceutically acceptable carrier or diluent to produce a pharmaceutical composition. Suitable carriers and diluents include any of those mentioned above. The composition may be formulated for parenteral, intramuscular, intravenous, subcutaneous, intraocular or transdermal administration.

The following Examples serve only to illustrate the invention and are not intended to limit the same.

### EXAMPLES

### Example 1

### Materials and Methods

Cell Culture and Oligonucleotide Treatment

The effect of antisense compounds on target nucleic acid expression can be tested in a variety of cell types. This can be routinely determined using Western Blot. The following cell types are provided for illustrative purposes, but other cell types can be routinely used.

### HEK Cells

Human embryonic keratinocytes (HEK) were obtained from the American tissue culture Collection. HEK cells were routinely maintained in DMEM:HAM (50:50) (Gibco) formulation.

Treatment with Antisense Compounds

Lipofectamine 2000™ (Invitrogen) or Genejuice™ (Novagan) was diluted in serum-free medium (dilution 40 μl/ml) and pre-incubated at room temperature for 5 minutes. An equal volume of oligonucleotide (diluted in serum-free DMEM/HAM) was then added and incubation continued for a further 15 minutes. Cells were washed with serum-free medium and 200 μl of the oligonucleotide mixture layered onto the cells together with a further 2001l serum-free DMEM/HAM. Cells were incubated at 37° C. in the presence of 5% CO₂ and medium replaced every 48 hours with fresh oligonucleotide in DMEM/HAM until the end of the experiment.

### EK4 Cells

EK4 cells are primary diploid human fibroblasts originally cultured from foreskin explant (Tyrell et al 1986). Cells were grown in 12 well plates and were maintained in MEM (Gibco) supplemented with 7.5% sodium bicarbonate and 15% heat inactivated serum.

Treatment with Antisense Compounds

Cells were transfected using Lipofectamine 2000™ or Lipofectin™ (Gibco). In experiments using Lipofectamine 2000™, the procedures were similar to that for HEK cells except that MEM was used instead of DMEM/HAM. In experiments using Lipofectin™, Lipofectin™ (diluted to 40 μl/ml) was mixed with an equal volume of oligonucleotide diluted in serum-free MEM, and the mixture incubated for 30 minutes at room temperature. 200 μl of the mixture was layered onto the cells together with further 200 μl MEM. At 8 hours, the medium was replaced with fresh MEM containing 0.25% BSA and the oligonucleotides at the appropriate concentrations. Incubation continued for a further 39 hours.

### 3T3 Cells

3T3 cells (American Type Culture Collection) were prepared and maintained as recommended by supplier. These cells showed comparable antisense-mediated PKB knockdown to that observed in HEK or EK4 cells, using the corresponding antisense sequences.

Treatment with Antisense Compounds

Lipofectamine 2000™ (diluted to 80 μl/ml with DMEM) was pre-incubated for 5 minutes at room temperature, and then incubated with appropriate dilutions of oligonucleotides at room temperature for 20 minutes. Cells were washed with DMEM before the addition of 200 μl of the oligonucleotide mixture together with a further 200 μl DMEM. Cells were incubated at 37° C. in the presence of 5% CO₂ and medium replaced every 48 hours with fresh oligonucleotide in DMEM (no addition) containing 0.25% bovine serum albumin, until the end of the experiment. Cells were treated with or without insulin and then extracted for Western blotting or PKB kinase assay, as appropriate

### Materials and Methods: Additional Procedures for Human Cancer Cells

### MCF-7 Human Breast Cancer Cells

MCF-7 human breast cancer cells (American Type Culture Collection) were routinely maintained in DMEM (Gibco) supplemented with 10% heat-inactivated foetal calf serum.

Treatment with Antisense Compounds

MCF-7 cells were transfected with Lipofectamine 2000™ (Gibco) using a protocol similar to that for 3T3 L1 cells. For experiments using cells in 12 well plates, Lipofectamine 2000™ (diluted to 80 μg/ml with DMEM) was pre-incubated at room temperature, and then incubated with appropriate dilutions of oligonucleotide for 20 minutes. 200 μl of the oligonucleotide mixture was added to the washed cells, together with a further 200 μl DMEM. The cells were then incubated at 37° C. and the medium replaced every 48 hours with fresh oligonucleotide in DMEM (no addition), until the end of the experiment. For experiments using cells in 24 well plates, the procedures were identical to that described above except that the total volumes used were decreased by a factor of 50%.

### SK-N-AS Human Neuroblastoma Cells

SK-N-AS neuroblastoma cells were obtained from the American Type Culture Collection and grown in 12 or 24 well plates using DMEM (Gibco) supplemented with 10% foetal calf serum.

Treatment with Antisense Compounds

SK-N-AS neuroblastoma cells were transfected with oligonucleotides and Lipofectamine 2000™ (Gibco) as for MCF-7 human breast cancer cells (see ABOVE). Oligonucleotide-containing medium was replaced at 48 hours and the cells were photographed, extracted and analysed after 72 hours.

### PC3 Human Prostate Cancer Cells.

PC3 human prostate cancer cells were maintained in RPMI medium (Cambrex) containing 10% foetal calf serum.

Treatment with Antisense Compounds

PC3 cells were transfected using Oligofectamine™ (Invitrogen). Oligofectamine™ (diluted to 80 μl/ml) was mixed with an equal volume of oligonucleotide diluted in serum-free RPMI, and the mixture incubated for 20 minutes at room temperature. 200 μl of the mixture was layered onto the cells together with further 200 μl RPMI. The medium was replaced every 48 hours with fresh RPMI and the oligonucleotides at the appropriate concentrations.

### Kelly Human Neuroblastoma Cells

Kelly human neuroblastoma cells were maintained according to supplier's instructions in DMEM supplemented with 10% foetal calf serum.

Treatment with Antisense Compounds

Kelly neuroblastoma cells were transfected with antisense oligonucleotides using Oligofectamine™ (Invitrogen), Lipofectamine 2000™ (Gibco) or GeneJuice™ (Novagen). Transfection reagent (diluted to 80 μl/ml) was mixed with oligonucleotide in DMEM (no additions) and incubated at room temperature for 20 minutes. 100 μl of the mixture was layered onto the cells in 24 well plates, together with a further 100 μl DMEM. The medium was replaced every 48 hours with fresh DMEM (no additions) containing oligonucleotides at the appropriate concentrations, until the end of the experiment.

### Western Blotting

Samples were separated by SDS gel electrophoresis and transferred to nitrocellulose. Membranes were blocked for one hour at room temperature in TBS-1% Tween 20 ™ (TBS-T) containing 5% milk fat protein (MFP) and then incubated overnight at 4° C. in primary antibody (1:1000 to 1:10000 dilution) in TBS-T containing 5% BSA (phospho-specific antibodies, ERK1/2 antibody) or 5% MFP (isoform-specific PKB antibodies, p70 S6Kinase). After this, the membranes were washed (4 times) with TBS-T, incubated with appropriate secondary antibody for 1 hour at room temperature in the presence of 5% MFP and the bands visualized by ECL and autoradiography. Bands were quantified by densistometric scanning and analyzed using Phoretix 1D software. All quantitative Western blot data for antisense knockdowns and other effects were obtained from band densities that fell within the sub-saturating linear range of the standard curve.

### PKB Kinase Assay

Immunoprecipitation of PKB from cell lysates (400 μl total volume, 0.15 mg protein) was carried out in the presence of immobilized PKBα G1 monoclonal antibody beads and lysis buffer for 2 hours at 4° C. The beads were then washed five times. The kinase assay was carried out at 30° C. for 30 minutes (assay conditions were linear) in the presence of kinase buffer, crosstide substrate (2 μg per reaction) and ATP (10 μCi [γ-32P]ATP and 200 pmol cold ATP per reaction). The reaction was terminated by spotting the kinase mixture onto P81 filter paper. The papers were washed repeatedly using 1% phosphoric acid. Incorporation of phosphate into crosstide was determined by scintillation counting.

### Results

### Antisense Oligonucleotides Knockdown PKB Isoforms Singly or in Combination.

The antisense compounds were analyzed for effect on PKB isoform protein levels by Western blot as described elsewhere. The results obtained are shown in Table 10 below and show that the antisense oligonucleotides effectively block expression of the intended PKB. Control mismatch oligonucleotides consisting of the antisense sequence with base changes along the length of the probe did not significantly effect the levels of all PKB isoforms.

TABLE 10
Inhibition of PKB isoforms by antisense oligonucleotides
singly or in combination with each other

| | | % Inhibition | % Inhibition | % Inhibition |
|---|---|---|---|---|
| Title of Antisense | | of PKBα | of PKBβ | of PKBγ |
| treatment | Oligonucleotide(s) | protein | protein | protein |
| Single AS(α) | SASα | 91 | ns | nd |
| Single AS(β) | SASβ | ns | 92 | nd |
| Single AS(γ) | SASγ | ns | ns | 96 |
| Double AS(α, β) | DASα + DASβ | 94 | 98 | ns |
| Triple AS(α, β, γ) | DASα + DASβ + | 93 | 94 | 94 |
| | SASγ | | | |

ns: not significant
nd: not determined

PKB Antisense Oligonucleotides Specifically Deplete their Target PKB Isoforms and not Other Key Kinases Upstream or Downstream of PKB.

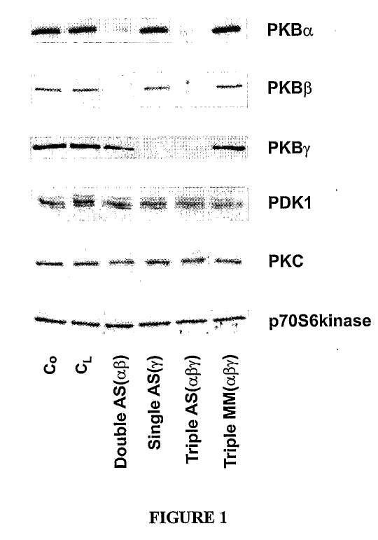

Cells were treated with or without oligonucleotides and cell lysates analysed by Western blotting for the indicated proteins, as described ABOVE. The results obtained are shown in FIG. 1. The results obtained show that the antisense oligonucleotides effectively and specifically inhibit their intended PKB or PKBs. Importantly, the oligonucleotides do not inhibit expression of kinases upstream or downstream of the intended PKB and also do not inhibit PKBs they are not targeted at.

### Dose Response Curve for the Depletion of PKB Isoforms by PKB Antisense Oligonucleotides.

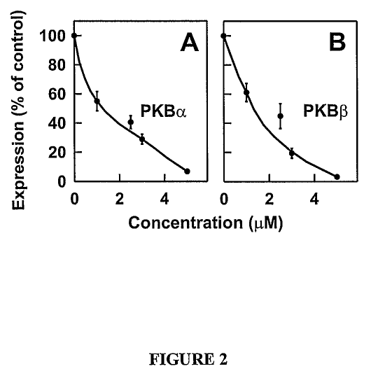

Cells were treated with Double AS(α,β) as described elsewhere. Cells were lysed and analysed for expression of (A) PKBα, (B) PKBβ. The results obtained are shown in FIG. 2. Results are expressed as % of that in control cells.

The results show effective inhibition of the intended PKB with almost complete inhibition above a concentration of 3 μM.

### Depletion of PKB Isoforms Using Antisense Oligonucleotides Inhibits Insulin-Stimulated Phosphorylation and Activation of Endogenous PKB.

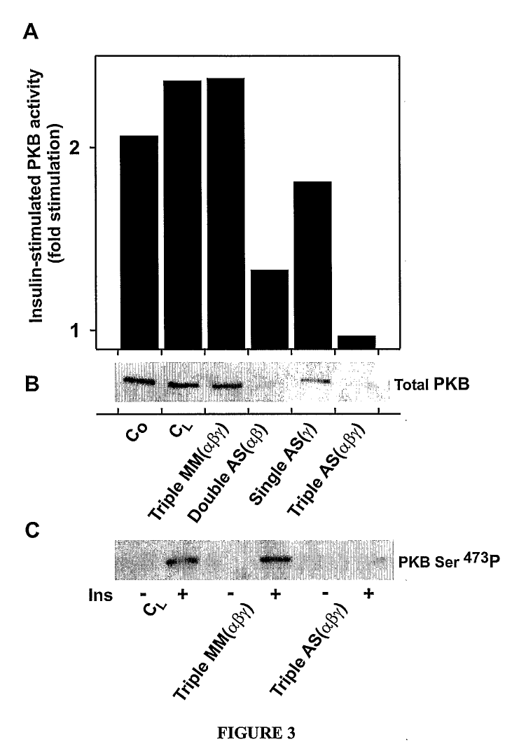

Cells were treated with or without oligonucleotides as shown and then stimulated with (+) or without (−) 100 nM insulin (Ins). The results obtained are shown in FIG. 3. Cell lysates were analysed for (A) PKB activity or were subjected to Western blot to determine (B) total PKB protein, or (C) phosphorylation of PKB at Ser473.

As can be seen, the single, double and treble antisense oligonucleotides progressively inhibit PKB activity and protein levels with the treble combination almost completely inhibiting both. The triple antisense combination almost entirely eliminated Ser473 phosphorylation. The results obtained demonstrating the potency of the antisense strategy and the sequences selected

### Depletions of PKB Isoforms Using Antisense Oligonucleotides Inhibits the Phosphorylation of Endogenous PKB Targets.

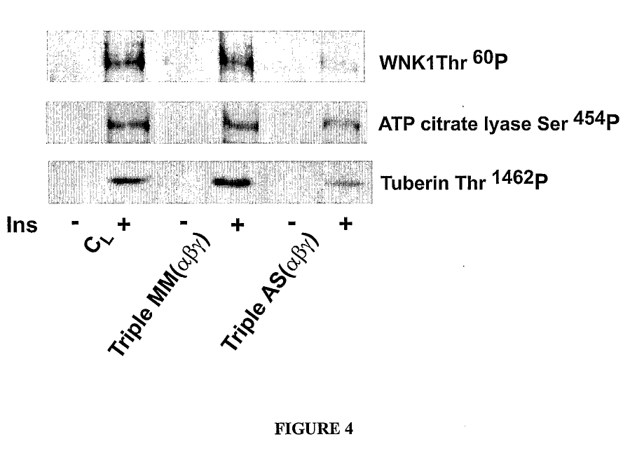

Cells were treated with or without oligonucleotides and then stimulated with (+) or without (−) 100 nM insulin (Ins) as described elsewhere herein. Cell lysates were centrifuged for 10,000 g for 10 minutes. The resultant supernatants were analysed by SDS gel electrophoresis. The results obtained are shown in FIG. 4. Results are Western blots showing phosphorylation of WNK-1 at Thr60; ATP citrate lyase at Ser454 or Tuberin at Thr1462,

The results obtained show that the triple antisense combination (Triple AS) substantially reduces insulin stimulated PKB mediated phosphorylation of WNK1Thr60, Tuberin Thr1462 and ATP citrate lyase Ser454.

### Example 2

### Antisense-Mediated Depletion of PKB with Triple Antisense Oligonucleotides Potently and Specifically Kills MCF-7 Human Breast Cancer Cells

MCF-7 cells were grown to near confluence and then treated with or without oligonucleotides, as described elsewhere. Photographs were taken at 120 h after starting treatment. Cells were treated with: (i) double antisense oligonucleotides against PKB α and β (DAS α,β—SEQ ID Nos: 4 and 22); (ii) triple antisense against PKB α, β and γ (Triple MM α, βγ—SEQ ID Nos: 4, 22 and 44); and (iii) triple α, β, γ mismatch control oligonucleotides (Triple MM α, βγ—SEQ ID Nos: 58, 59 and 57). Controls with (i) C_{O}, control untreated cells; and (ii) C_{L}, Lipofectamine-only treated cells were also performed.

The results obtained showed substantial cancer cell death with DAS a, P and Triple AS α, β, γ. Mismatch control oligonucleotides had no effect, as did the Lipofectamine control.

### Time Course of Death of MCF-7 Human Breast Cancer Cells by PKB Antisense Oligonucleotide Treatment.

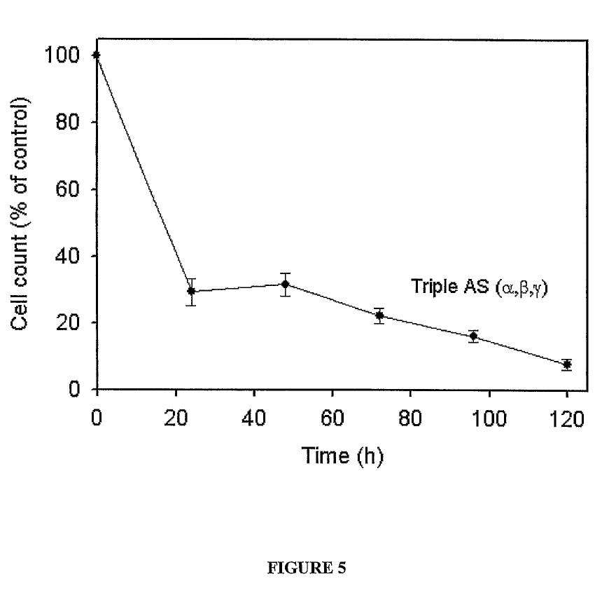

MCF-7 cells were treated with Triple AS(α,β,β) or Double AS(α,β) as described above with a Lipofectamine alone control also being performed. Photographs of cells following treatment were taken at 0, 24, 48, 72, 96 and 120 hours post-treatment. Both antisense treatments produced rapid and almost complete cell death. FIG. 5 shows the time course of viable cell number for triple antisense treated cells, as expressed as a percentage of that for C_{L} Lipofectamine-only treated control cells determined at the same time point.

### PKB Antisense Oligonucleotide Treatment Rapidly and Potently Kills SK-N-AS Human Neuroblastoma Cells.

SK-N-AS neuroblastoma cells at near confluence were treated with or without PKB Triple AS(α,β,γ) with Lipofectamine 2000™ as a transfection reagent, as described above. A control of lipofectamine-only treated cells and a separate control of cells treated with no oligonucleotides or Lipofectamine were also performed. Photographs were taken at 72 h post-transfection. The photographs showed that the Triple AS (α,β,γ) almost entirely killed the cells, whilst the control cells were unaffected.

### PKB Antisense-Mediated Death of Kelly Human Neuroblastoma Cells is Optimal Using Oligofectamine.

Kelly neuroblastoma cells were transfected with or without PKB antisense oligonucleotides using Oligofectamine™, Lipofectamine 2000™ or GeneJuice™ as transfection reagent. Cultures were transfected with Triple AS (α,β,γ) or Triple MM (α,β,γ) and compared to controls treated with the transfection agent alone. Photographs of the viable cells at 96 h post-transfection were taken.

Oligofectamine transfection gave a much higher level of cancer cell death with Triple AS (α,β,γ) than either other transfection agent. The transfection agent only and mismatch controls showed the effect was due to the antisense oligonucleotides, rather than any direct effect of the transfection agent or the introduction of any oligonucleotide. The results show that Oligofectamine™ was the optimal transfection reagent and in particular is highly effective for Neuroblastoma cells.

Time Course of the Death Kelly Human Neuroblastoma Cells with PKB Triple Antisense Oligonucleotide Treatment.

Kelly human neuroblastoma cells were treated with or without PKB triple antisense oligonucleotides, Triple AS (α,β,γ), using Oligofectamine™ as transfection reagent, as discussed above. Photographs were taken at 0, 24, 48, 72 and 96 hours and showed rapid cell death, with cell death clearly visible at 24 hours and progressively increasing over time.

### PKB Antisense Oligonucleotides, but not Control Oligonucleotides, Cause Significant Death of PC3 Human Prostate Cancer Cells.

PC3 human prostate cancer cells were treated with or without PKB triple antisense oligonucleotides or mismatch oligonucleotides using Oligofectamine™ as transfection reagent, as described elsewhere. Samples were treated with: (i) nothing; (ii) oligofectamine alone; (iii) Triple AS (α,β,γ); or (iv) Triple MM (α,β,γ). Photographs were taken at 168 h post-transfection and showed almost complete cell killing with the Triple AS (α,β,γ) in comparison to the controls.

### Additional Isoform-Specific Antisense Oligonucleotides Deplete PKB, Induce Breast Cancer Cell Death, and Inhibit the Phosphorylation of Key Downstream Protein Targets.

MCF cells were treated with the additional a isoform-targeted PKB antisense oligonucleotides as indicated (also see Tables 1) using methods described elsewhere. After 96 h treatment, photographs were taken and these showed all of the oligonucleotides as capable of inducing cell death.

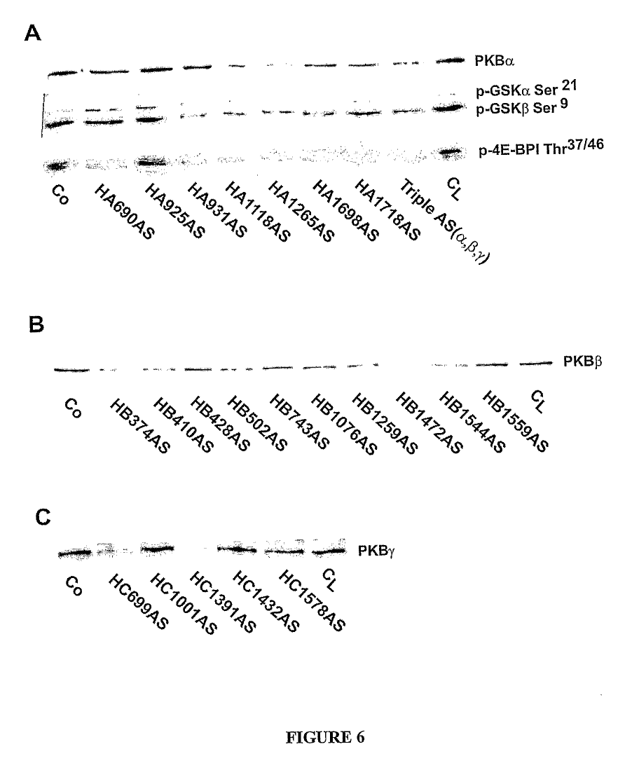

Cell lysates were then prepared by centrifugation at 10,000 g for 10 minutes and the resultant supernatants analysed by SDS gel electrophoresis. The results are shown in FIG. 6A. The Western blots show PKBα protein; the phosphorylation of GSK-3α at Ser²¹; GSK-3β at Ser⁹; and 4E-BPI at Thr^{37/46}. The results show the order of descending potency is HA1265, HA1118AS, HA1718AS, HA1698AS, HA931AS, HA690AS and HA925AS.

In a separate experiment, breast cancer cells were treated with β isoform-targeted PKB antisense oligonucleotides (see Table 2) and the resultant Western blot showing PKBβ protein after 96 h treatment is shown in FIG. 6B. The descending order of potency is HB1472AS, HB374AS, HB1259AS, HB410AS, HB1544AS, HB502AS, HB1076AS, HB743AS, HB428AS and HB1559.

In a further experiment, breast cancer cells were treated with γ isoform targeted PKB antisense oligonucleotides (see Table 3) and the resultant Western blot showing PKBγ protein after 96 hours treatment is shown in FIG. 6C. The descending order of potency is HC1391AS, HC699AS, HC1578AS, HC1432 and HC1001.

1. An antisense compound 8 to 30 nucleotides in length which is capable of inhibiting wholly or partially the activity of Protein Kinase B (PKB) isoforms a, P and/or y, the antisense compound comprising the sequence of:
(i) any one of sequences of SEQ ID Nos: 1 to 74;
(i) the equivalent region to the sequence of SEQ ID Nos: 1 to 74 in a non-human mammalian PKB; or
(ii) a sequence with at least 80% sequence identity to the sequence of (i) or (ii).

2. An antisense compound according to claim 1 comprising the sequence of:
(i) any one of SEQ ID Nos 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72 or 74;
(ii) the equivalent region to a sequence of (i) in a non-human mammalian PKB; or
(iii) a sequence with at least 80% sequence identity to the sequence of (i) or (ii).

3. An antisense compound according to claim 1, wherein the compound is 28 or less nucleotides in length.

4. An antisense compound according to claim 3, wherein the compound is 22 or less nucleotides in length.

5. An antisense compound according to claim 4 which consists of the sequence of
(i) any one of sequences of SEQ ID Nos: 1 to 74 or;
(ii) a sequence with 70% to any of the sequences of (i).

6. An antisense compound according to claim 1 wherein the compounds is selected from:

| | |
|---|---|
| SASα - Single AS(α) | |
| GCCTGCGCTCGCTGTCCA | (SEQ ID No. 2) |
| SASβ - Single AS(β) | |
| CTCGCGGATGCTGGCCGA | (SEQ ID No. 20) |
| SASγ - Single AS(γ) | |
| GGCCCCACCAGTCTACTG | (SEQ ID No. 44) |
| DASα | |
| TGACCACGCCCAGCCCCC | (SEQ ID No. 4) |
| DASβ | |
| TGACCACACCCAGCCCCC | (SEQ ID No. 22) |

7. An antisense compound according to claim 6 which is SASβ, DASα or DASβ.

8. An antisense compound according to claim 1, which comprises the sequence of any one of SEQ ID Nos: 14, 12, 18, 4, 38, 24, 36, 26, 40, 30, 22, 50, 46 and 44.

9. An antisense compound according to claim 1 wherein the compound is an antisense oligonucleotide.

10. An antisense compound according to claim 1 wherein the antisense oligonucleotide comprises at least one modified internucleoside linkage.

11. A combination of at least two different antisense compounds collectively capable of inhibiting wholly or partially at least two of PKB α, β and γ.

12. A combination according to claim 11 wherein the combination is collectively capable of inhibiting wholly or partially all of PKB α, β and γ.

13. A combination comprising an antisense compound according to claim 1 and at least one other antisense compound.

14. A combination according to claim 12, wherein at least one of said other antisense compounds wholly or partially inhibits Protein Kinase B (PKB) isoforms α, β and/or γ.

15. A combination according to a claim 13 which comprises:
(i) a combination of oligonucleotides collectively capable of inhibiting PKB α and β wholly or partially;
(ii) a combination of oligonucleotides collectively capable of inhibiting PKB α and γ wholly or partially;
(iii) a combination of oligonucleotides collectively capable of inhibiting PKB β and γ wholly or partially; and/or
(iv) a combination of oligonucleotides collectively capable of inhibiting PKB α, β and γ wholly or partially

16. A combination according to claim 15 which comprises at least two oligonucleotides collectively capable of inhibiting PKB α, β and γ wholly or partially.

17. A combination comprising:
(i) an antisense compound according to claim 1; and
(ii) a therapeutic agent other than (i).

18. A combination according to claim 17 wherein the therapeutic agent of (ii) is selected from an anticancer agent, an agent for treating diabetes, an agent for treating a degenerative neurological disorder and an agent for treating a mental disorder.

19. A polynucleotide capable of expressing an antisense compound according to claim 1.

20. A polynucleotide according to claim 19 which is a vector.

21. A host cell comprising a polynucleotide according to claim 19.

22-23. (canceled)

24. Method of treating cancer, diabetes, a degenerative neurological condition and/or a mental disorder which comprises administering an effective amount of an antisense compound according to claim 1 to a subject with such a disorder.

25. A method according to claim 24 wherein the degenerative neurological condition is selected from Huntington's disease and Alzheimer's and/or the mental disorder is selected from bipolar disorder and schizophrenia.

26. A method according to claim 24 wherein one or two or, but not all of, PKB α, β and γ are selectively inhibited wholly or partially.

27. A method of inhibiting wholly or partially the expression of PKB α, β and/or γ in cells or tissues comprising contacting said cells or tissues with an antisense compound according to claim 1.

28. A method according to claim 27 wherein the the cells or tissue are human.
